(19) United States
(12) Patent Application Publication
(10) Pub. No.: US 2004/0152066 A1
(43) Pub. Date: Aug. 5, 2004

(54) Freeze-tolerant eukaryotic cells

(76) Inventors: An Tanghe, Lovenjoel (BE); Johan Thevelein, Blanden (BE); Patrick Van Dijck, Zichem (BE)

Correspondence Address: MUSERLIAN AND LUCAS AND MERCANTI, LLP, 475 PARK AVENUE SOUTH, NEW YORK, NY, 10016, US

(21) Appl. No.: 10/476,728

(22) PCT Filed: May 3, 2002
(86) PCT No.: PCT/EP02/04943

(30) Foreign Application Priority Data
May 3, 2001 (EP) 01201626.7

(51) Int. Cl. A01N 1/02; C12N 15/85; C12N 15/82; C12N 15/74

(52) U.S. Cl. 435/2; 435/455; 435/468; 435/483

(57) ABSTRACT

The present invention relates to the use of proteins facilitating water diffusion or water transport through the cell membrane, preferably aquaporin or aquaporin related proteins to obtain freeze-tolerant eukaryotic cells, preferably yeast cells or plant cells. It relates further to a method for obtaining such cells, and to freeze-tolerant cells, characterized by an enhanced expression level of proteins facilitating water diffusion or water transport through the cell membrane.

The present invention relates to the use of proteins facilitating water diffusion or water transport through the cell membrane, preferably aquaporin or aquaporin related proteins to obtain chilling and/or freeze-tolerant eukaryotic cells, preferably yeast cells or plant cells. It relates further to a method for obtaining such cells, and to chilling and/or freeze-tolerant cells, characterized by an enhanced expression level of proteins facilitating water diffusion or water transport through the cell membrane. Freeze-damage is an important problem in eukaryotic cells that occurs when eukaryotic cells are placed—for storage, or by environmental conditions—at temperatures below 0° C. Freeze-damage can occur, amongst others, in plants, during cold nights, in sperm cells that are stored frozen before their use for fertilisation, or in yeast, especially in cases where frozen doughs are prepared. Especially in the case of yeast and plants, there is a need for freeze-tolerant cells, to avoid freeze-damage.

Bread making is one of the oldest food-manufacturing processes and depends on the fermentative capacity of baker's yeast Saccharomyces cerevisiae for the rising of the dough. For each type of dough (plain dough, sweet dough, sour dough) the selection, isolation or construction of yeast strains bearing the appropriate characteristics is required. The same is true for a more recent application of baker's yeast with a high potential for widespread use: frozen dough (Attfield, 1997).

Although offering convenience, automation and economy of scale to bakers, this method suffers from an inherent and as yet unresolved drawback: the reduced leavening capacity of the dough after storage in frozen form due to a low survival and concomitant loss of fermentation capacity of the yeast.

Although the production conditions for bakers' yeast have been optimized in order to obtain yeast with a high stress resistance, the initiation of fermentation is associated with a rapid drop in freeze-resistance (Merrit, 1960). This is apparently due to activation of several nutrient-in particular sugar-controlled signal transduction pathways such as the Ras-cAMP pathway and the FGM pathway (Thevelein, 1994, Park et al., 1997, Thevelein and de Winde, 1999, Van Dijck et al., 1995).

Neither the addition of more yeast or protective additives nor the optimalisation of processing conditions has resulted in a satisfying solution for the loss of rising capacity of the dough during frozen storage (Kline and Sugihara, 1968, Neyreneuf and Van Der Plaat, 1991). The availability of yeast strains that are deficient in the nutrient induced loss of stress-resistance and show the same performance for all other industrially relevant properties would be of large economic benefit for the production of frozen doughs (Randez-Gil et al., 1999).

Up to now, some yeast strains with improved freeze-resistance have been isolated from natural sources, selected from culture collections or constructed by hybridisation or mutation (Oda et al., 1986, 1993, Hino et al., 1987, Hahn and Kawai, 1990, Nakagawa and Ouchi, 1994, Almeida and Pais, 1996, Van Dijck et al., 2000, EP0967280). Upon characterisation of these strains some correlations between freeze-resistance and specific cellular components have been reported. In addition to the protective effect of a high trehalose level (Gélinas et al., 1989, Hino et al., 1990, Attfield et al., 1992, Iwahashi et al., 1995, Lewis et al., 1997, Kim et al., 1996, Diniz-Mendes et al., 1999, Shima et al., 1999) and an enhanced expression of ‘heat shock’ proteins (Komatsu et al., 1990, Kaul et al., 1992), freeze-resistance seems to be correlated to a certain extent with a particular lipid composition of the plasma membrane (Murakami et al., 1996), an efficient respiratory metabolism (Lewis et al., 1993), the accumulation of charged amino acids (Takagi et al., 1997), the capacity to restore damage to actin and the enzymes of glycolysis (Hatano et al., 1996) and the activity of the cytoplasmic Cu, Zn superoxide dismutase (Park et al., 1998). However, up to now it has not been possible to improve freeze-resistance in yeast by targeted modification of any one of these factors or of any other factor. Also there is no precise knowledge concerning the mechanism by which these factors would contribute to the improvement of freeze-resistance nor is any specific gene known of which reduced or enhanced expression improves freeze-tolerance of yeast. Hence, at present it is not possible to construct in a controlled way yeast strains with improved freeze-tolerance by modification of the expression of one or more endogenous yeast genes.

When plants are cooled down to around or below 0° C., they risk to suffer from freeze-damage. Lower temperatures, especially frost, may cause plant cells to freeze-destroying intracellular structures, causing death or severe damage to the plants. Several methods have been proposed to avoid chilling and freeze-damage in plants, including active protection from frost, as well as selection of resistant cultivars. Active methods are mostly based on heating or spraying of warm water, or utilization of oil in water emulsions. These methods are rather expensive and labour intensive, and require a continuous monitoring of the outside temperature. Selection of cold resistant varieties has been described (Bolduc et al., 1985) but the success rate of classical breeding techniques has been limited. EP0891702 describes the construction of temperature tolerant and freeze-tolerant plants, by transforming the plant with a vector carrying a gene encoding choline oxidase. This method has the advantage that the plant itself becomes freeze-tolerant, and no active treatment is needed in case of frost. However, this result was obtained using a bacterial choline oxidase, which may affect other commercially important properties of the plant or may be unwanted because of its bacterial origin.

Similar to freeze-resistance in yeast, chilling resistance in plants seems to be correlated to a modified lipid composition in the plant membrane. U.S. Pat. No. 5,614,393 describes the use of microbial δ-6-desaturases to obtain a high γ-linolenic acid content in plants. WO9213082 describes the use of Arabidopsis thaliana glycerol-3-phosphate acyltransferase to modify the fatty acid content of phosphatidylglycerols in transgenic tobacco. Because of the limited successes with these approaches, it is clear that there is a need for more powerful methods conferring chilling and freeze-resistance to eukaryotic cells, especially yeast cells and plant cells, preferably by overexpression of endogenous genes. Surprisingly, we found that proteins that facilitate water diffusion or transport through the cell membranes, such as aquaporin and aquaporin-like proteins can confer freeze-resistance to eukaryotic cells.

Aquaporins have been identified in nearly all life forms; they belong to a highly conserved family of membrane proteins called the MIP (major intrinsic protein) family, with molecular masses between 26 and 30 kDa. In plants, a distinction is made between aquaporins present in the plasma membrane (PIPs) and those present in the tonoplast (TIPs). Like the other members of the MIP family, aquaporins typically contain six membrane-spanning domains, with the N- and C-termini both located on the cytoplasmic side of the membrane. They contain between the second and the third, and between the fifth and the sixth membrane-spanning domain, hydrophilic loops comprising a highly conserved asparagine-proline-alanine motif. One can make a further distinction between real aquaporins, which are supposed to be involved only in water transport, and aquaporin-like molecules such as aquaglyceroporins, that may transport other small molecules such as glycerol besides water. Members of the aquaporin family are, as a non-limiting example, Aqy1 and Aqy2 in Saccharomyces cerevisiae, γTIP and PIP1a in Arabidopsis thaliana and hAQP1 in humans. Aquaporin-like molecules comprise, amongst others, Fps1 and YFL054C (homologue) in S. cerevisiae.

It is generally accepted that aquaporins function as narrow pores through which water molecules flow passively down their concentration gradient (Tyerman et al., 1999). In plants, aquaporins are assumed to play a role in osmotic adjustment (Maurel, 1997), hydraulic conductivity (Johansson et al., 2000) and in cell expansion (Chaumont et al., 1998; Balk and de Boer, 1999). Contrary to our results, Li et al. (2000) suggest that repression of the rice aquaporin RWC1 gene may improve water stress-induced chilling tolerance. However, the repression observed was probably due to the osmotic stress caused by the high mannitol concentration added in the growth medium.

The role of aquaporins and the fysiological relevance of aquaporin-mediated water transport in S. cerevisiae are not clear yet. This yeast possesses two genes encoding aquaporins, which are polymorphic, leading to important differences between different strains. Strain Σ1278b contains two functional alleles (AQY1-1 and AQY2-1) whereas most other laboratory strains do contain the apparently inactive allele AQY1-2, which cannot mediate water transport in an oocyte system, and the allele AQY2-2, which has a frameshift mutation (Meyrial et al., 2001). However, the absence of functional alleles does not seem to affect neither the growth nor the viability of the strains. On the contrary, Bonhivers et al. (1998) showed that deletion of AQY1-1 results in a significantly improved viability when cultures of the mutant were subjected to cycles of hyper- and hypo-osmotic stress. Meyrial et al. (2001) suggest that AQY2-1 may play a role during cell expansion.

In addition to aquaporins and other members of the MIP family, other types of proteins can also be involved in facilitating water diffusion or transport through the cell membrane. Such proteins are membrane proteins, involved in transport of other compounds, such as the cystic fybrosis gene product (Hasegawa et al., 1992), facilitative glucose transporters (Fischbarg et al., 1990; Loike et al., 1993; Fischbarg and Vera, 1995) or sodium-glucose co-transporters (Loike et al., 1996), but it may also be regulatory proteins, that control the rate of the diffusion or transport, without being a part of a membrane channel. Indeed it is known that the activity of several transporter molecules, including proteins facilitating water diffusion or transport through the cell membrane, such as aquaporins, are regulated by phosphorylation: TPK2 is involved in water homeostasis in yeast (Robertson et al., 2000), the aquaporin PM28A of spinach is activated by phosphorylation (Johansson et al., 1998) and in a similar way, α-TIP is activated by phosphorylation through protein kinase A (Maurel et al., 1995).

Up to now, no indication has been presented that proteins facilitating water diffusion or transport through the cell membrane, such as aquaporin or aquaporin-like proteins, may be involved in chilling and/or freeze-tolerance in yeast.

A first aspect of the invention is the use of proteins facilitating water diffusion or transport through the cell membrane to obtain chilling and/or freeze-tolerance in a eukaryotic cell. As mentioned above, said protein facilitating water diffusion or transport may be directly involved in water transport, or it may be a regulatory protein that controls the rate of the diffusion or transport, without being a part of a membrane channel. Preferably, said protein is used to obtain freeze tolerance, even more preferably, said protein is used to obtain tolerance against fast freezing. Preferably, said protein is an aquaporin or an aquaporin-like protein, and said eukaryotic cell is a plant cell or a yeast cell, more preferably a Saccharomyces, Schizosaccharomyces or Candida cell, most preferably a Saccharomyces cerevisiae cell. Preferably, said Saccharomyces cerevisiae cell is a baker's yeast cell. When the coding sequence is placed downstream an appropriate promoter sequence, endogeneous as well as non-endogenous aquaporins may be used, as well as aquaporins with different cellular locations (e.g. PIPs and TIPs in plants). A preferred embodiment is the use of an aquaporin or an aquaporin-like protein comprising SEQ ID N°2, SEQ ID N° 4 or SEQ ID N° 6.

Another aspect of the invention is a method to obtain chilling and/or freeze-tolerance in a eukaryotic cell, comprising a) placing a gene encoding a protein facilitating water diffusion or transport through the cell membrane downstream a promoter sequence suitable for expressing said gene in said eukaryotic cell, b) transforming or transfecting the nucleic acid comprising said promoter and gene into said eukaryotic cell and c) growing said eukaryotic cells under conditions suitable for the expression of said gene.

Preferably, said method is a method to obtain freeze-tolerance, even more preferably, said method is a method to obtain tolerance against fast freezing. Preferentially said protein facilitating water diffusion or transport through the cell membrane is an aquaporin or an aquaporin-like protein.

Suitable promoters are known to the person skilled in the art. The endogenous promoter of an aquaporin gene may be considered as a suitable promoter, especially when a multi-copy vector is used. Preferably, said promoter is a constitutive promoter, or a promoter with optimal expression under the growth conditions used. Preferably, said eukaryotic cell is a plant cell, or a yeast cell, preferably said yeast cell is a Saccharomyces, Schizosaccharomyces or Candida cell, more preferrably said yeast cell is a Saccaromyces cerevisiae cell. Preferably, said Saccharomyces cerevisiae cell is a bakers yeast cell. Vectors for transferring recombinant sequences into eukaryotic cells are known to the person skilled in the art and include, but are not limited to self-replicating vectors, integrative vectors, artificial chromosomes, Agrobacterium based transformation vectors and viral vector systems such as retroviral vectors, adenoviral vectors or lentiviral vectors.

Transformation and transfection methods for eukaryotic cells are also known to the person skilled in the art and include, but are not limited to protoplast transformation, chemical treatment of the cells, electroporation, particle gun mediated transformation, Agrobacterium mediated transformation and virus mediated transformation.

A preferred embodiment is said method, whereby said protein facilitating water diffusion or transport through the cell membrane comprises SEQ ID N°1, SEQ ID N°3 or SEQ ID N°5.

Alternatively, said method may be carried out by inserting a non-endogenous promoter upstream of a gene encoding a protein facilitating water diffusion or water transport throught the cell membrane. Non-endogenous promoter as used here comprises both promoters is derived from another gene from the same organism as well as promoters derived from a related or non-related gene from another organism. Preferably the 5′ upstream sequence of an endogenous gene encoding a protein facilitating water diffusion or transport through the cell membrane is replaced by a constitutive promoter or a promoter with optimal expression under the growth conditions used. This method is especially useful when said endogenous gene is not or not sufficiently active under the growth conditions used.

Another aspect of the invention is a chilling and/or freeze-tolerant eukaryotic cell, preferably a freeze-tolerant eukaryotic cell, more preferably an eukaryotic cell resistant to fast freezing, whereby said eukaryotic cell is characterized by an enhanced expression of a protein facilitating water diffusion or transport through the cell membrane. Preferentially, said protein is an aquaporin or an aquaporin-like protein. Indeed, it is known that, for yeast, such as S. cerevisiae Σ1278b, certain growth conditions such as the shift from a medium with 0.5 M KCl to a hypo-osmotic medium without KCl can induce AQY2 expression (Meyrial et al, 2001). On the other hand, compounds that directly enhance aquaporin expression, such as chlorophenylthio-cAMP have been described (Matsumura et al, 1997); it is known indeed that certain aquaporin promoters do comprise a cAMP-responsive element and compounds, activating said response element are known to the person skilled in the art. Preferably, said eukaryotic cell, characterized by an enhanced expression of an aquaporin or an aquaporin-like protein, is obtained by the method according to the invention. Preferably, said eukaryotic cell is a plant cell or a yeast cell, more preferably a Saccharomyces, Schizosaccharomyces or Candida cell, most preferably a Saccharomyces cerevisiae cell. Preferably, said Saccharomyces cerevisiae cell is a baker's yeast cell.

The quantification of the expression of proteins facilitating water diffusion or transport through the cell membrane is depending upon the nature of the protein. For transmembrane proteins, as aquaporins, the proteins can be quantified by—as a non-limiting example—the use of specific, fluorescently labeled antibodies, and quantification of the fluorescent label per cell, by the use of FACS.

Still another aspect of the invention is the use of compounds, which activate a protein facilitating water diffusion or transport through the cell membrane, such as an aquaporin or an aquaporin-like protein, to obtain chilling and/or freeze-tolerance, preferably freeze-tolerance, even more preferably tolerance against fast freezing, in an eukaryotic cell. Preferably, said eukaryotic cell is a yeast cell or a plant cell. Even more preferably, said yeast cell is a Saccharomyces, Shizzosaccharomyces or Candida cell. Most preferably, said yeast cell is a Saccharomyces cerevisiae cell. Said compounds are, as a non-limiting example, protein kinases such as protein kinase A. Overexpression of said kinases will lead to activation of the aquaporins, and will result in freeze-tolerance. Moreover, it is known that cAMP antagonists such as 8-bromoadenosine 3′,5′-cyclic monophosphate, forskolin or 3-isobutyl-1-methylxanthine are stimulating protein kinase A and result in an activation of α-TIP (Maurel et al, 1995). As a consequence, said compounds may also be used to obtain freeze-tolerance. In a similar way, inactivation of phosphatases that deactivate the phosphorylated proteins facilitating water diffusion or transport through the cell membrane, such as aquaporins, can be used to activate said proteins, resulting in freeze-tolerance of the cell in which said proteins are activated. Compounds that inhibit the phosphatase activity will have a similar effect. Said compounds are known to the person skilled in the art.

Another aspect of the invention is the use of a chilling and/or freeze-tolerant baker's yeast according to the invention to prepare frozen dough.

Still another aspect of the invention is a dough, comprising at least one yeast cell according to the invention.

Still another aspect of the invention is a plant, comprising at least one freeze-tolerant plant cell according to the invention. Preferably, said freeze-tolerant plant cell is obtained by a method according to the invention. Indeed, as a non-limiting example, a plant cell, transformed to overexpress aquaporin may be regenerated to result in a plant that overexpresses aquaporin either systemically, or only in well-defined tissues, depending on the promoter used. Methods to regenerate plants from a single plant cell are known to the person skilled in the art, as well as suitable promoters for systemic or tissue specific expression. Said plants comprising at least one freeze-tolerant plant cell according to the invention are more freeze tolerant, and will be more resistant to chilling and freeze-damage, especially to damage caused by frost. Especially those tissues, which are sensitive to frost, like the tissues in blossoms, may be targets for overexpression of one or more proteins facilitating water diffusion or water transport through the cell membrane.

Methods to detect yeast cells and plant cells, according to the invention, when they are embedded in respectively a dough or a whole plant, are know to the person skilled in the art and include, but are not limited too, PCR techniques and immunological techniques.

### Definitions

Gene as used herein refers to a polymeric form of nucleotides of any length, either ribonucleotides or deoxyribonucleotides. This term refers only to the primary structure of the molecule. The term includes double- and single-stranded DNA and RNA. It also includes known types of modifications, for example methylation, “caps” substitution of one or more of the naturally occurring nucleotides with an analogue. It includes, but is not limited to, the coding sequence, and may include non-translated intron sequences. However, as used here, the promoter sequence is not included; this sequence will be referred as “endogenous promoter” when it indicates the promoter naturally occuring upstream of the gene.

Endogenous gene means that the gene is naturally occuring in the wild type organism.

Plant cell as used here does not necessarily indicate an individual plant cell, but may be one or more cells of a plant up to a total plant. In that case, the expression of the aquaporin or aquaporin like protein may be limited to one or more parts or organelles of the plant, or it may be expressed in the whole plant.

Chilling damage as used here is the damage caused by placing the eukaryotic cells, as individual cells or as organisms, for a shorter or longer time at temperatures between 4 and 15° C. Freezing damage is the damage caused by placing the eukaryotic cells, as individual cells or as organisms, for a shorter or longer time at temperatures below 4° C., normally at temperatures below 0° C. Tolerance against fast freezing as used here means tolerance against freezing conditions in which intracellular ice crystallization is occuring. Situations in which fast freezing may occur are, amongst other, lyophilisation of cultures of all kinds of eukaryotic cells, as well as frost, preferably night frost for plants and plant cells.

Chilling- or freeze-tolerant cells are cells that show significantly less chilling or freezing damage after a chilling or freeze period than a non-transformed (in case of chilling- or freeze-tolerant cells obtained by transformation) or non-mutated (in case of chilling- or freeze-tolerant cells obtained by mutation) reference, which is cultured in standard conditions before the treatment. Said non-transformed or non-mutated cells will be referred as wild type strains. Standard culture conditions are dependent upon the type of eukaryotic cells; these conditions are known to the person skilled in the art. For yeast, as an example, standard culture conditions are growth in YPD at 30° C. till stationary growth phase.

Enhanced expression as used here is an expression that is significantly higher than for the corresponding control cell. For mutants and transformants, the control is the corresponding non-mutated or non-transformed cell, grown in the same conditions as the mutant or transformed cell. For wild type cells, grown under special conditions, the same type of cell grown under standard conditions is used as control. In the case of cells, obtained by crossing, or by sporulation and crossing, the control cells are both parental cells. The expression can be measured either at the level of mRNA, e.g. by Northern hybridization, but preferably at the protein level, e.g. by specific antibodies. Growth conditions indicate the general conditions (such as temperature, pH, medium composition, oxygen supply . . . ) in which the cell is kept. It does not necessarily imply that the cell is growing under those conditions: the cell may be metabolic active without cell division.

A protein facilitating water diffusion or water transport through the cell membrane includes every protein that has a positive effect on passive water diffusion or active water transport through the membrane. Said protein may be part of a protein complex, comprising one or more subunits. The protein may be a structural protein, such as a water channel, or a regulatory protein, such as a protein envolved in the control of the opening or closing of the channel.

Compound as used here means any chemical or biological compound, including simple or complex inorganic or organic molecules, peptides, pseudo-mimetics, proteins, antibodies, carbohydrates, nucleic acids and derivatives thereof.

### BRIEF DESCRIPTION OF THE FIGURES

FIG. 1. Differential expression of ORFs YLL052C and YLL053C between freeze-resistant baker's yeast strains HAT36, HAT43, HAT44 and freeze-sensitive baker's yeast strain SS1 at the onset of fermentation, as detected by Yeast Index Genefilters (Research Genetics) and Pathways (Research Genetics). Expression values were normalised against all data points. ACT1 was used as internal reference.

FIG. 2. Differential expression of AQY2 between freeze-resistant baker's yeast strains HAT36, HAT43 and freeze-sensitive baker's yeast strain SS1 at the onset of fermentation, as confirmed by Northern analysis using YLL052C+YLL053C as a probe. Expression values were normalised against ACT1-expression signals.

FIG. 3. Initial glucose consumption (IGC), glucose consumption after freezing (FGC) and relative glucose consumption (RGC) of resp. Saccharomyces cerevisiae strain BY4743 (A) and Σ1278b (B) wild type strain, AQY1-1 overexpression strain, AQY2-1 overexpression strain and two control strains having integrated resp. an empty vector (integrative plasmid pYX012 KanMX containing TPI promotor) and a vector with the disrupted AQY2-2 allele. The cells were frozen (FGC) or cooled (IGC) at the onset of fermentation (40 min after the addition of 100 mM glucose to stationary phase cells). After thawing, glucose consumption was measured during 3 h (A) resp. 4 h (B). RGC is calculated as (FGC/IGC)×100.

FIG. 4. Diagnostic restriction analysis of PCR amplified genes AQY1 and AQY2 from S. cerevisiae BY4743 (1) in comparison to ‘non-functional’ alleles AQY1-2 and AQY2-2 from W303-1A (3) and ‘functional’ alleles AQY1-1 and AQY2-1 from S. cerevisiae Σ1278b (2), showing strain BY4743 does not carry a functional allele, neither for AQY1 nor for AQY2. Restriction analysis was performed as described in Laizé et al., 2000.

FIG. 5. Growth curves (Bioscreen measurements) in (A) YPD-, (B) YPM- and (C) molasses-medium of S. cerevisiae BY4743, AT25 and S47 wild type strains and AQY2-1 overexpression strains, showing no obvious growth defects upon overexpression of the water channel.

FIG. 6. Initial glucose consumption (IGC), glucose consumption after freezing (FGC) and relative glucose consumption (RGC) of the original industrial baker's yeast strain AT25, the AQY1-1 overexpression strain, the AQY2-1 overexpression strain and two control strains having integrated resp. an empty vector (integrative plasmid pYX012 KanMX containing TPI promotor) and a vector with the disrupted AQY2-2 allele. The cells were frozen (RGC) or cooled (IGC) at the onset of fermentation (30 min after the addition of 100 mM glucose to stationary phase cells). After thawing, glucose consumption was measured during 2.5 h. RGC is calculated as for FIG. 3.

FIG. 7. Diagnostic restriction analysis of PCR amplified genes AQY1 and AQY2 from the industrial baker's yeast strains AT25 (1) and S47 (2) (resp. pools of different alleles) in comparison to ‘non-functional’ alleles AQY1-2 and AQY2-2 from W303-1A (3) and ‘functional’ alleles AQY1-1 and AQY2-1 from Σ1278b (4), showing strains AT25 and S47 don't not carry any functional AQY2 but do posses at least one functional AQY1-1 allele. Restriction analysis was performed as described in Laizé et al., 2000.

FIG. 8. Initial glucose consumption (IGC), glucose consumption after freezing (FGC) and relative glucose consumption (RGC) of S. cerevisiae strain BY4743 overexpressing the wild type hAQP1 resp. the mutant hAQP1 from plasmid pYeDP1/8-10 (under the control of the inducible GAL10-CYC1 hybrid promotor and the PGK terminator) in comparison to a control strain transformed with an empty plasmid. The cells were frozen (RGC) or cooled (IGC) at the onset of fermentation (40 min after the addition of 100 mM glucose to stationary phase cells). After thawing, glucose consumption was measured during 4 h. YPD-grown cells (first three double bars, prefix ‘d’) as well as YPG-grown cells (last three double bars, prefix ‘g’) were tested. Calculation of RGC was as for FIG. 3.

FIG. 9. Initial glucose consumption (IGC), glucose consumption after freezing (FGC) and relative glucose consumption (RGC) of wild type S. cerevisiae Σ1278b strain, aqy1 deletion strain, aqy2 deletion strain and aqy1aqy2 deletion strain in Σ1278b background for non-fermented (A) and fermented (B) cells. The cells were frozen (RGC) or cooled (IGC) at the onset of fermentation (40 min after the addition of 100 mM glucose to stationary phase cells). After thawing, glucose consumption was measured during 4 h. Calculation of RGC was as for FIG. 3.

FIG. 10: Strategy used to obtain a marker-free integration.

FIG. 11: Localisation of the primers used to check the marker-free integration.

FIG. 12: Initial glucose consumption (IGC), glucose consumption after freezing (FGC) and relative glucose consumption (RGC) of the original industiral baker's yeast strain AT25, the AQY2-1 overexpression strain (integrative plasmid pYX012 KanMX containing TPI promotor) and the AQY2-1 overexpression strain selected without the usage of the resistance marker (AT25+AQY2-1w/o R; two independent cultures). The cells were frozen (RGC) or cooled (IGC) at the onset of fermentation (30 min after the addition of 100 mM glucose to stationary phase cells). After thawing, glucose consumption was measured during 2.5 h. Two independent experiments were performed (A and B).

FIG. 13: Freeze tolerance of RD28 and AQY2-1 overexpression Schizz. pombe strains in comparison with a control strain (empty plasmid). Late exponential phase cells were frozen for 1 hour at −30° C. Survival of frozen cells compared to non-frozen cells (cooled on ice) is expressed as % CFU. (R=repressive conditions NMT1-promotor, I=non-repressive conditions NMT1-promotor).

FIG. 14: Growth of RD28 and AQY2-1 overexpression Schizz. pombe strains in comparison with a control strain (empty plasmid) in EMM lacking thiamine (A) and EMM containing thiamine (B). Bioscreen measurements, readings are saturated at OD₆₀₀-values above 1.2.

FIG. 15: Western analysis of RD28 (lanes 3 and 4) and AQY2-1 overexpression Schizz. pombe strains (lanes 6 and 7) in comparison with a control strain (empty plasmid) (lanes 2 and 5), in repressive (lanes 2, 3, 6) and non-repressive (lanes 4, 5, 7) conditions of the NMT1-promotor. 10 μl TriChromRanger™ (Pierce) was loaded as molecular weight marker (lane 1).

FIG. 16: Freeze tolerance of heterozygous (aqy1Δ) and homozygous (aqy1ΔΔ) C. albicans AQY1 deletion strains. Cells were grown overnight in YPD (stationary phase) and uracil-deficient minimal medium (exponential phase) and were frozen for 1 hour and 1 day. Survival of frozen cells compared to non-frozen cells (cooled on ice) is expressed as % CFU.

FIG. 17: Growth of heterozygous (aqy1Δ) and homozygous (aqy1ΔΔ) C. albicans AQY1 deletion strains in YPD and uracil-deficient minimal medium (Bioscreen measurements, readings are saturated at OD₆₀₀-values above 1,2).

FIG. 18: Resistance of industrial baker's yeast AT25 aquaporin overexpression strains against slow and fast freezing. Strains were grown in laboratory conditions and cell suspensions were frozen in three different ways. Left panel: cells rapidly frozen in liquid nitrogen (RF). Middle panel: cells rapidly frozen at −30° C. by immersion in a methanol bath. Right panel: cells slowly cooled from 0° C. till −30° C. (SF). Additionally, cells were thawed in three different ways: rapidly in a warm water bath at 30° C. (wwb), intermediately at room temperature (air), slowly on ice (ice).

FIG. 19: Survival in small doughs upon slow freezing and storage. Baker's yeast AT25: cultured in laboratory conditions and harvested from liquid medium. Baker's yeast LAT25: cultured in industrial conditions and resuspended from pressed yeast cake.

FIG. 20: Freeze tolerance of tobacco BY-2 cells measured after 15 min at −30° C. The results are expressed as factor increase in cell death, as compared with a control, kept on ice. AQY2-1: BY-2 cells transformed with the S. cerevisiae gene AQY2-1. RD28: BY-2 cells, transformed with the A. thaliana gene RD28.

### EXAMPLES

### Materials and Methods to the Examples

Yeast Strains and Culture Conditions.

The yeast strains used in this study are listed in Table 1. Cells were routinely grown in YP (1% (w/v) yeast extract (Merck), 2% (w/v) bactopepton (Oxoid)) with 2% glucose (YPD), 2% galactose (YPG) or 0.5% molasses (YPM) at 30° C. in an orbital shaker or were plated on YPD or YPM media containing 1.5% agar. Selection for geneticin resistance was made with YPD liquid media or plates supplemented with 150 mg/liter of G418 sulfate (Life Technologies). Strains grown under industrial conditions were grown and processed in a baker's yeast pilot plant.

Strains with an Industrial Background.

Starting from the industrial yeast strain S47 (Lesaffre Développement) different mutants were isolated that are deficient in ‘fermentation induced loss of stress resistance’ (‘fil’ mutants) in conditions that resemble commercial dough preparation (Van Dijck et al., 2000, EP0967280). Besides the improved freeze-resistance, several mutants displayed a growth rate and fermentation capacity comparable to the original strain. The strain AT25 also performed better for freeze-resistance after growth in pilot scale conditions. S47 and AT25 were sporulated and mutual mating of freeze-resistant spores of AT25 and freeze-sensitive spores of S47 resulted in the hybrid strains HAT36, HAT43, HAT44 and SS1 respectively. Integration of pYX012 KanMX/AQY1-1, pYX012 KanMX/AQY2-1 and pYX012 KanMX/YLL052-053C at the TPI-locus resulted in geneticin resistant strain of AT25 overexpressing resp. AQY1-1, AQY2-1 and AQY2-2. Also pYX012 KanMX was inserted in this strain. All strains were checked by diagnostic PCR using genomic DNA as template.

Strains with a Laboratory Background.

In strains 10560-6B (Σ1278b-derivative strain) and BY4743 (S288C-derivative strain) integration of pYX012 KanMX/AQY1-1, pYX012 KanMX/AQY2-1 and pYX012 KanMX/YLL052-053C at the TPI-locus resulted respectively in geneticin resistant strains overexpressing AQY1-1, AQY2-1 and AQY2-2. Also pYX012 KanMX was inserted in both strains. All strains were checked by diagnostic PCR using genomic DNA as template. Deletion strains of AQY1-1, AQY2-1 or both genes together in the 10560-6B strain background (strain Σ1278b in which auxotrophic markers have been introduced) were kindly provided by Stefan Hohmann.

Plasmids and Primers.

The plasmids and primers used in this study are listed in Table 1. The basic vector used for all overexpression constructs is the integrative plasmid pYX012 (Novagen) containing a TPI promotor and a URA3 selective marker. Plasmid pYX012 was modified with a dominant marker for use in prototrophic strains by cloning the EcoRV/PvuII-fragment containing the loxP-KanamycinMX-loxP cassette from pUG6 in pYX012 digested in the URA3 marker with Stul. AQY2-1 was subcloned in pYX012 KanMX from pYX242/AQY2-1 (kindly provided by Vincent Laizé) using restriction enzymes EcoRI and BamHI. AQY1-1 and AQY2-2 were amplified by PCR using genomic DNA of resp. the 10560-6B and W303-1A strains as template and using primer pairs ANT108, ANT109 and ANT106, ANT107. The resulting fragments were cut with EcoRI, HindIII and EcoRI, XmaI resp. and cloned into pYX012 KanMX digested with the same restriction enzyme combinations. Plasmids pYeDP-CHIP (Laizé et al., 1995) and pYeDP-CHIPmut were kindly provided by S. Hohmann. pYeDP-CHIPmut is identical to pYeDP-CHIP, except for a mutation in the CHIP28 water channel gene leading to a A73M conversion in the protein, which inactivated its function.

Genomic DNA Extraction.

The following was added to pelleted cells: 300 μl TE, 300 μl PCI and glass beads. The celts were broken in a Fastprep dissicator during 20 s at speed 5 m/s. The tubes were centrifugated during 10 min at 13000 rpm and supernatant was taken off in a clean Eppendorf tube.

PCR Amplifications.

The primers used in this study are listed in Table 1. The PCR-reactions generating fragments for cloning in plasmids or for integration in genomes were all done using the Expand High Fidelity system (Boehringer Mannheim) with 10×buffer 2 containing 15 mM MgCl₂. Reactions contained 300 μM primers, 200 μM dNTP's, 1×buffer 2, 50 ng of DNA template and 0.75 μl polymerase. For amplification of AQY1 (primers ANT 108 and ANT 109) and AQY2 (primers ANT 110 and ANT 111) using 1 μg genomic DNA of AT25, S47, BY4743, W303-1A or Σ1278b as template, 30 cycles were performed in following conditions (after an initial denaturation step of 2 min at 94° C.): denaturation for 30 s. at 94° C., annealing for 30 s. at 50° C., elongation for 1 min at 72° C. To complete the final strand, the last step was allowed to run 10 min at 72° C. (Laizé et al., 2000). Correct incorporation of integrative plasmids in the genome was checked on 1 μl genomic DNA using primer TPIprom-FW (inside construct) and ANT 117 (flanking construct) in combination with KanRW, ANT107, ANT109 or ANT111 (for empty vector, AQY2-2, AQY1-1 and AQY2-1 constructs respectively). For amplification of TPIpromotor+AQY2-1 using NcoI-digested pYX012/KanMX AQY2-1 as template, following program was used: denaturation for 4 min at 94° C., 10 cycles: denaturation for 15 s. at 94° C., annealing for 30 s. at 55° C., elongation for 1.5 min at 72° C., 10 cycles: denaturation for 15 s. at 94° C., annealing for 30 s. at 55° C., elongation at 72° C. for 1.5 min and each cycle 5 s in addition. To complete the final strand, the last step was allowed to run 10 min at 72° C. Primers used were EANT1 and EANT2, consisting of 60 bp complementary to flanking regions of YLL052C/YLL053C and 20 bp complementary to the TPIpromotor+AQY2-1 fragment. Correct incorporation of the fragment in the genome was checked on 1 μl genomic DNA using primer combinations TPIprom-FW+ANT 114 (downstream of construct) and TPIprom-RW+ANT115 (upstream of construct).

Restriction Analysis.

PCR amplification of AQY1- and AQY2-alleles followed by diagnostic restriction analysis was performed as described in Laizé et al., 2000. Strains W303-1A and 10560-6B were used as reference-strains for the amplification and analysis of AQY1-2, AQY2-2 and AQY1-1, AQY2-1 alleles respectively.

RNA Isolation

Strains were grown till stationary phase in YPD or YPM at 30° C. in an orbital shaker. Cells were collected and resuspended in YP. After 30 min of incubation, glucose was added to a final concentration of 100 mM. Culture samples for total RNA isolation were taken 30 min after the resuspension of cells in YP and 30 min after the addition of glucose and were immediately added to 30 ml of ice-cold water. The cells were washed once with ice-cold water and stored at −70° C. Total RNA was isolated using RNApure™ Reagent (GeneHunter® Corporation) according to manufacturers instructions.

Microarray Analysis.

Microarray analysis was performed using Yeast Index Genefilters® (Research Genetics) according to manufacturers instructions. Probes were prepared by RT-PCR in the presence of alpha ³³P-dCTP using total RNA as template. The filter comparisons were made using Pathways™ 2.0 software (Research Genetics).

Northern Analysis.

Total RNA was separated in formaldehyde-containing agarose gels and transferred to nylon membranes. Probes used for hybridisation were ³²P-labelled fragments generated with Highprime (Boehringer Mannheim). Actin was used as loading standard. Signals were quantified using a phosphorimager (Fuji, BAS-1000; software, MacBAS V2.5) and expressed as % of the actin messenger level.

Yeast Transformation.

50 ml YPD was inocculated with 1.5 ml of overnight pre-culture and grown under vigorous shaking for 4 h to 6 h at 30° C. Cells were collected by centrifugation (5 min, 1500 rpm) and supernatant was removed. Cells were resuspended in 1 ml 0.1M LiAc, the suspension was transferred to an eppendorf tube and centrifuged for 2 min at 2000 rpm. Supernatant was removed, cells were resuspended in 100 to 800 μl 0.1M LiAc and put at roomtemperature for 10 min The following was added to a new tube: 50 μl cells, 5 to 10 μl of purified PCR product, 300 μl PLi and 5 μl ssDNA. Suspensions were vortexed for 10 s. and incubated at 42° C. for 30 min Cells were collected (4000 rpm, 1 min) supernatant was removed, cells were washed in 1 ml H₂O and resuspended in 1 ml YPD. In case of prototrophic markers, cells were incubated at 30° C. for 3 h to 4 h, plated on selective plates, and incubated at 30° C. In case of auxotrophic markers, cells were plated immediately.

Growth Curves.

The onset of growth and the maximum growth rate was determined via automatic OD₆₀₀-measurements using the Bioscreen apparatus (Labsystems). The following parameters were programmed: 250 μl each well, 30 s shaking per min (medium intensity), OD₆₀₀ measurement each 30 min. At OD₆₀₀ 1.2 the measuring system is saturated. Therefore also cultures of 50 ml were inocculated and samples were taken manually.

Residual Glucose Consumption After Freezing and Freeze-Drying.

Strains were grown till stationary phase in YPD or YPM at 30° C. in an orbital shaker. Equal amounts of cells (corresponding to an OD₆₀₀ of 20 for laboratory strains and an OD₆₀₀ of 15 for industrial strains) were collected and resuspended in YP. After incubation at 30° C. for 30 min cell suspensions were divided in equal amounts. The first sample was immediately put in ice water. To the second sample glucose was added till a final concentration of 100 mM and cell suspensions were incubated at 30° C. for 30 min (industrial strains) or 40 min (laboratory strains) and immediately put in ice water. After harvesting and resuspending in pre-cooled YP, both samples were divided and placed in two conditions: kept on ice on the one hand and frozen on the other hand.

After freezing (ethanol bath at −30° C.) and storage during one day (freezer at −30° C.) glucose was added till a final concentration of 30 mM to control samples and after thawing to the samples that were frozen. After either 3 or 4 hours of incubation at 30° C., cell suspensions were centrifuged and the glucose concentration of the supernatant was determined using Trinder reagens (Sigma Diagnostics). The residual glucose consumption (RGC) was calculated as the glucose consumption of frozen samples (FGC) compared to control samples (IGC) from both fermenting and non-fermenting cells and expressed as percentage [RGC=(FGC/IGC)×100].

To test resistance against freeze-drying, essentially the same procedure was followed. In this case two extra aliquots (40 μl) were frozen (ethanol bath at −30° C.), kept at −30° C. in a freezer for one day and exposed to freeze-drying stress during two hours (LyolabA, LSL Secfroid). Special care was taken that no thawing occurred during the whole process by maintaining the samples in a freezing block during freezing and freeze-drying. After freeze-drying, culture-containing Eppendorf tubes were reconstituted by adding an adequate volume of YP.

Frozen Doughs.

100 μl of an overnight culture in 3 ml YPD was spread out on molasses plates (25 ml) and grown at 30° C. during 24 hours. In a falcontube 7.5 g flour and 0.15 g of salt were weighed. Molasses plates were washed with 6 ml water resulting in a 5.5 ml cell suspension. Exactly the same amount of each strain was added to the flour and salt (usually about 5 g). The dough was mixed and kneaded using a spatula, divided in 0.25 g (0.24-0.26 g) amounts in fastprep tubes, centrifugated for 15 min at 13000 rpm and fermented for 30 min at 30° C. in the oven. All doughs were put at −30° C. in the cryostate for 1 hour except for 2 controls (non-frozen). Part of the doughs was stored in the freezer (−30° C.), part of the doughs were put in the cryostate and subjected to freeze/thaw cycles (30° C./−30° C./30° C. in 2 hours). For each measuring point (x freeze/thaw cycles or y days in the freezer) 2 tubes for each strain were taken out of the cryostate or freezer, 1 ml TS and 0.5 ml glass beads were added to the dough which then was vortexed for 1 min to release the yeast cells from the dough. The obtained suspension was then diluted and plated on YPD.

### Example 1

### AQY1 and AQY2 are Differentially Expressed Between Different Freeze-Resistant and Freeze-Sensitive Industrial Baker's Yeast Strains

Using nylon membranes representing all ORFs of S. cerevisiae the expression pattern of freeze-resistant strains AT25, HAT36, HAT43 and HAT44 in comparison to freeze-sensitive strains S47 and SS1 was studied. SS1 is a derivative strain from production strain S47. HAT36, HAT43, HAT44 are derivative strains from strain AT25, a freeze-resistant mutant of S47 that was isolated as a strain displaying a clear ‘fil’-phenotype (deficient in fermentation induced loss of stress resistance) (Tanghe et al., 2000; EP0967280).

Expression patterns at the onset of fermentation, i.e. 30 min after the addition of glucose to stationary phase cells were studied, because of the ressemblance with industrial frozen dough production where the freezing of the dough is preceded by a pre-fermentation period of about 30 min (Merrit, 1960, Attfield et al., 1997, Randez-Gil et al., 1999).

Several genes were identified as differentially expressed (ratio 3 or more) in at least 2 comparisons of a resistant and sensitive strain: 67 genes were upregulated, 15 genes were downregulated in the resistant strains as compared with the sensitive strains. Only 8 genes showed an at least 3-fold differential expression in each of the comparisons; these differences were confirmed by Northern analysis with the same and with independent batches of total RNA. For some of these genes, single overexpression (in S47 and AT25) and deletion (in BY4743) resulted in a minor effect on stress-resistance.

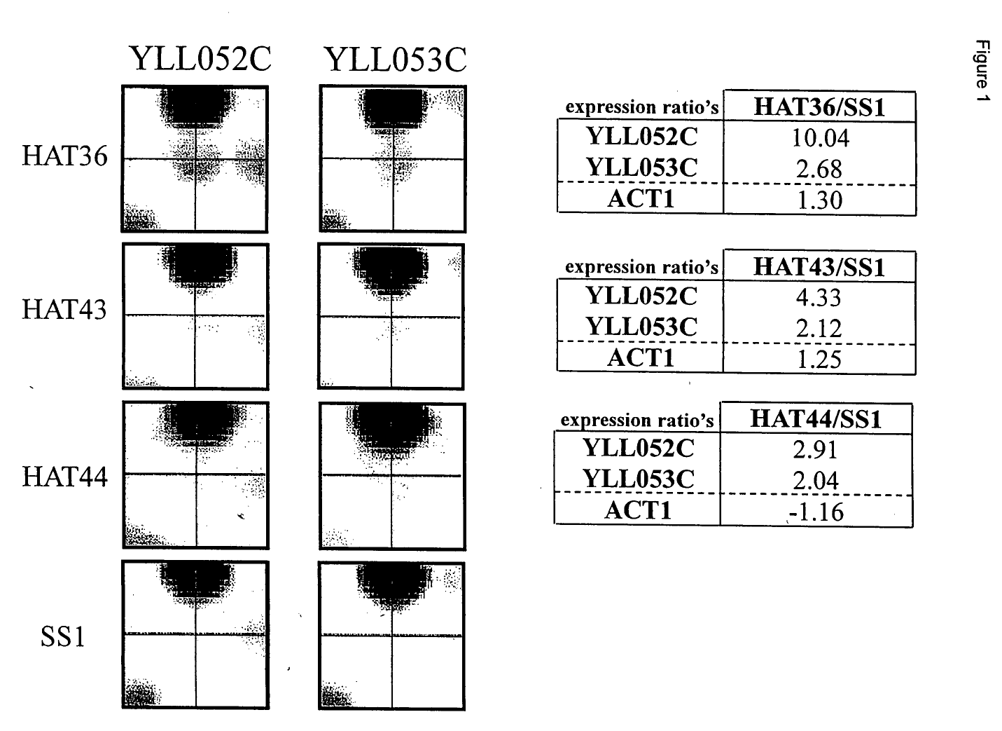

ORFs YLL052C and YLL053C were upregulated in some of the freeze-resistant strains (FIG. 1). In most laboratory strains, industrial strains and natural isolates they are overlapping. Only in Σ1278b these ORFs form an intact ORF encoding a functional AQY2 water channel (Laizé et al., 2000, Carbrey et al., 2001a, Meryal et al., 2001). Expression of AQY1 (YPR192W), a second gene in the genome of S. cerevisiae encoding a water channel, could not be monitored during micro-array analysis since this ORF is not represented on the filters. In most laboratory strains AQY1 encodes a non-functional water channel. In strain Σ1278b and most industrial strains and natural isolates this ORF forms encodes a functional water channel (Laizé et al., 2000, Carbrey et al., 2001a, Meryal et al., 2001). Because of the large homology of AQY1 and AQY2 (75.5% at the DNA level), cross hybridisation during micro-array analysis is unlikely but cannot be excluded (Rep et al., 2000). Although the differences in expression observed for AQY2 were not the most pronounced ones, the possible connection between upregulation of a water channel and improvement of freeze-resistance was striking.

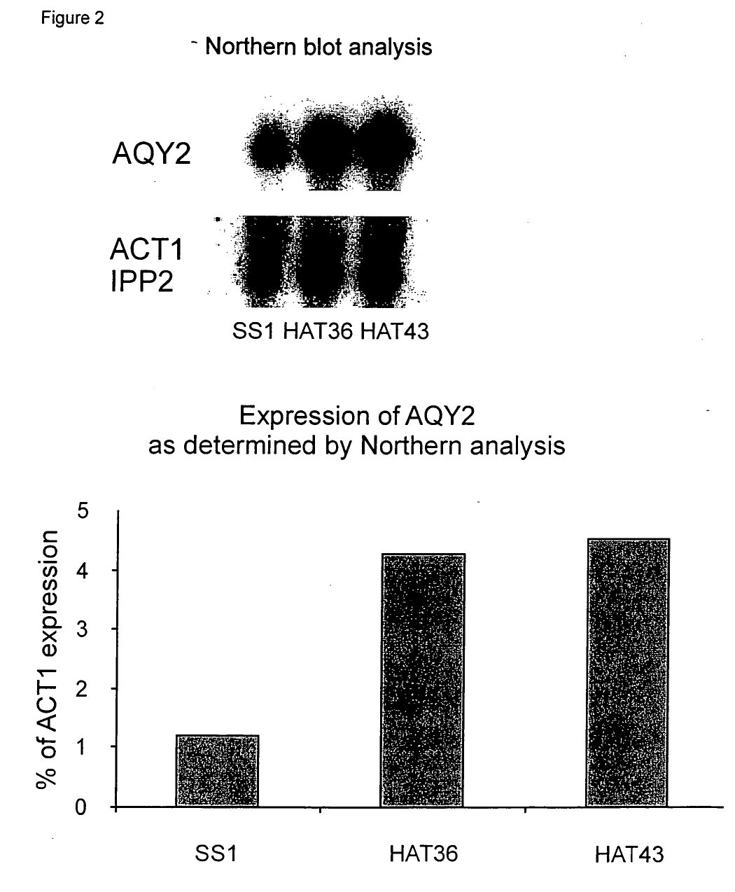

For confirmation of differential expression by Northern analysis, more specific probes were designed to check the expression patterns of AQY1 and AQY2 separately. The probes were tested using deletion strains in the Σ1278b-background and overexpression strains in the BY4743-background. In the condition used for micro-array analysis, AQY1 is not expressed in neither the sensitive nor the resistant strains, whereas AQY2 shows a higher expression level in resistant strains compared to sensitive strains (FIG. 2). In addition, Northern analysis was performed during a so called ‘glucose shift’ of AT25 and S47: total RNA was isolated in stationary phase, 30 min after resuspension in YP, 30 min and 90 min after subsequent addition of 100 mM glucose. In both strains, AQY1 seems to be induced 30 min after resuspension of stationary phase cells in YP (for YPD grown cells: higher levels for AT25 compared to S47, for YPM grown cells: higher levels for S47 compared to AT25) and repressed again 30 min after addition of glucose. On the contrary, AQY2 seems to be induced upon the addition of glucose (higher levels for AT25 compared to S47, for YPD as well as YPM grown cells). The same patterns of induction and repression were found using laboratory strain Σ1278b.

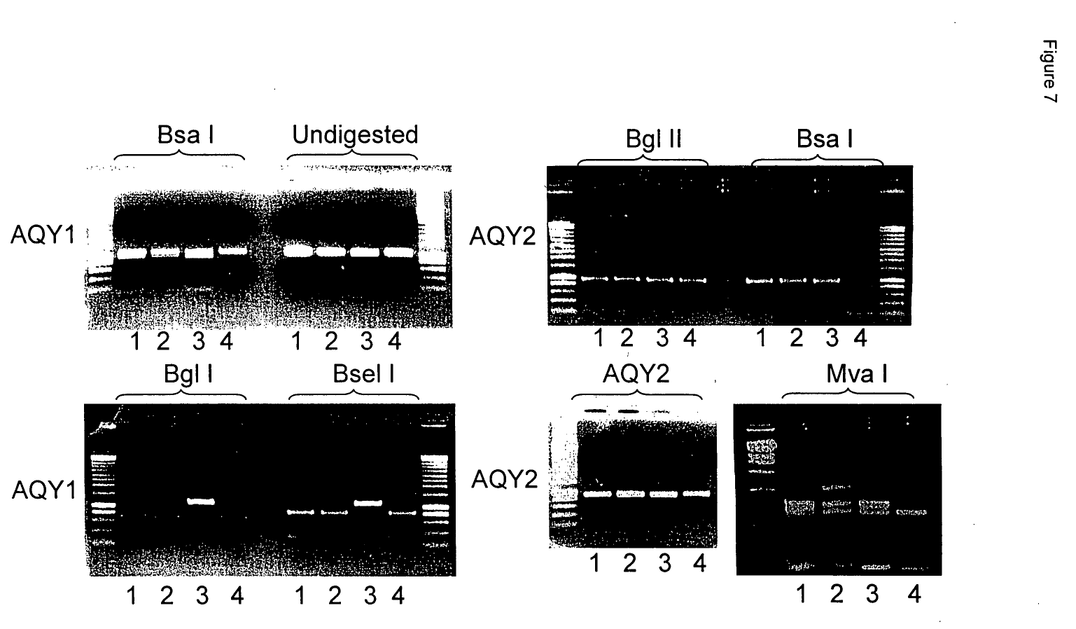

Restriction analysis shows the absence of a functional allele of AQY2 in AT25 and S47 (FIG. 7), rendering a relationship between higher expression of AQY2 30 min after glucose addition and higher freeze-resistance of AT25 at the onset of fermentation unlikely. However, from the restriction analysis it can not be excluded that a particular AQY2-allele in these strains encodes a functional water channel.

Restriction analysis shows the presence of both functional and non-functional alleles of AQY1 in AT25 and S47 (FIG. 7). Possibly, higher protein levels of the water channel AQY1 (resulting from the higher level of mRNA's 30 min after resuspension of stationary phase cells in YP) are protecting the cells upon freezing (30 min after subsequent addition of 100 mM glucose). For YPD grown cells, levels in AT25 tend to be higher compared to S47 in this condition. Contradictory, for YPM grown cells levels in S47 tend to be higher compared to AT25 in this condition.

### Example 2

### Overexpression of Functional Alleles AQY1-1 and AQY2-1 Improves Freeze-Resistance in Both Laboratory and Industrial Saccharomyces cerevisiae Strains Without Affecting Growth

Aquaporin encoding alleles AQY1-1 and AQY2-1 from strain Σ1278b were overexpressed, in two laboratory strains (BY4743 and Σ1278b) and in two industrial strains (AT25 and S47). It has been shown that AQY1-1 mediates water transport when expressed in Xenopus laevis oocytes (Bonhivers et al., 1998, Laizé et al., 1999). Using stopped-flow analysis, it has also been demonstrated that AQY2-1 acts as a water transporter (Meyrial et al., 2001).

Laboratory Strains.

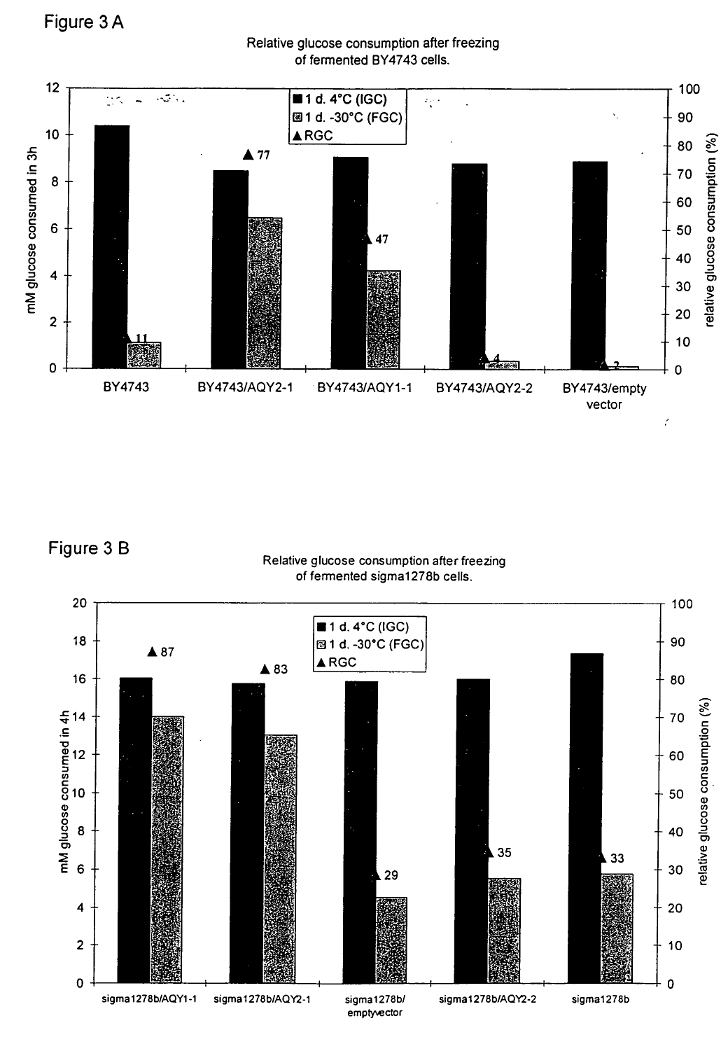

BY4743 and Σ1278b strains overexpressing AQY1-1 and AQY2-1 clearly showed an improved relative glucose consumption after pre-fermentation and freezing, compared to the wild type strain and two control strains that resp. have integrated an empty vector or a vector with the non-functional AQY2-2 allele (FIG. 3A and B). The effect was also monitored in non-fermented cells (prior to freezing). The improvement of freeze-resistance is not due to a difference in initial glucose consumption since IGC-values are comparable for all strains. The improvement of freeze-resistance is also not due to the presence of the vector since RGC-values for wild type cells as such and wild type cells containing an empty plasmid do not significantly differ. The effect is also observed when cells are frozen for several days or when cells are submitted to freeze/thaw cycles before freezing.

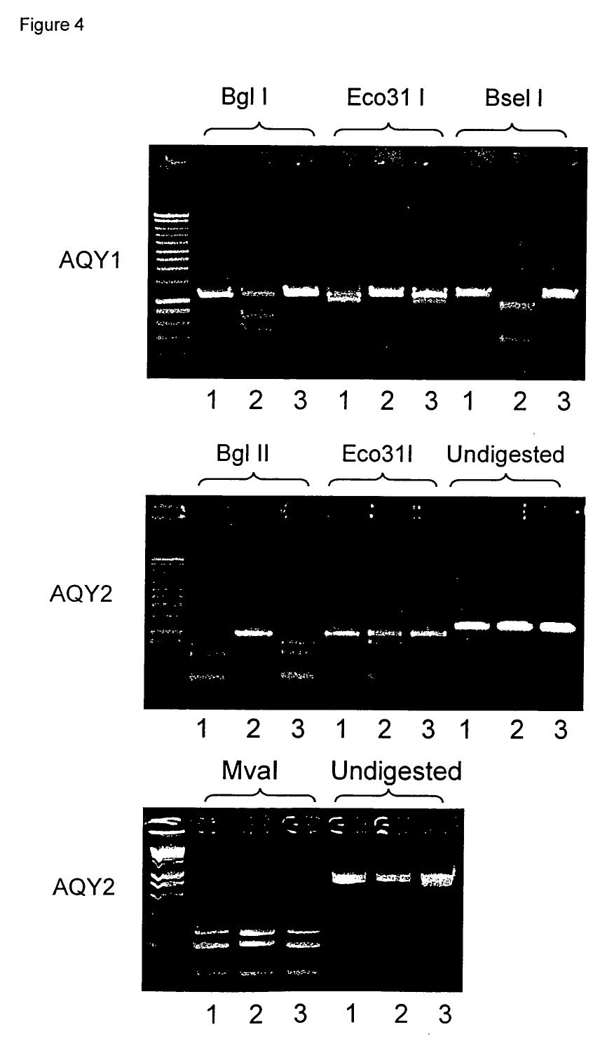

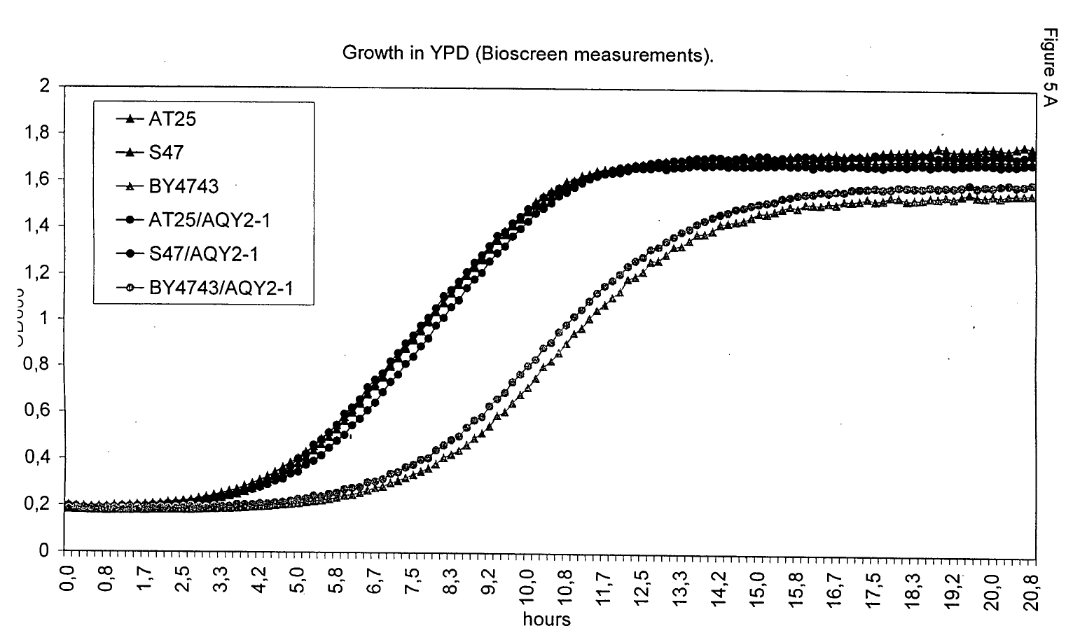

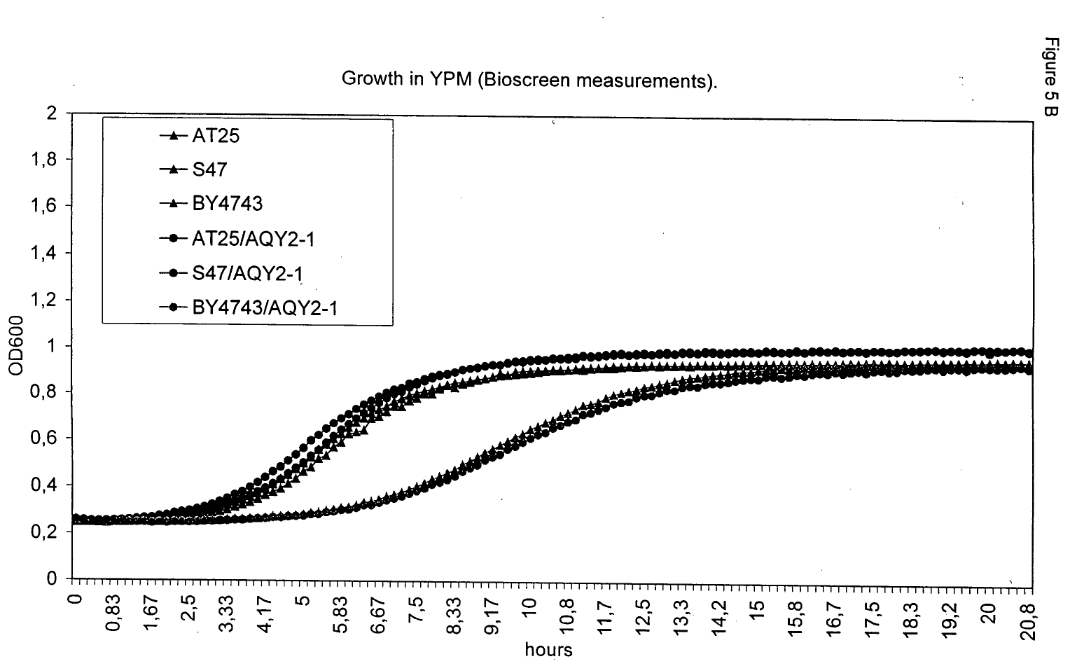

As shown by diagnostic restriction analysis, BY4743 does not carry a functional allele, neither for AQY1 nor for AQY2 (FIG. 4). This is in accordance with published results since BY4743 is a S288C-derivative (Laizé et al., 2000). Diagnostic restriction analysis also shows that Σ1278b carries functional alleles of both water channels. This is in accordance with published results (Laizé et al., 2000). The levels of relative glucose consumption are higher for wild type Σ1278b compared to wild type BY4743. Growth curves (Bioscreen measurements) of the strains did not reveal any obvious growth defect resulting from overexpression of either of both water channels in strain BY4743, neither for growth in YPD, nor YPM, nor molasses (FIG. 5).

Industrial Strains.

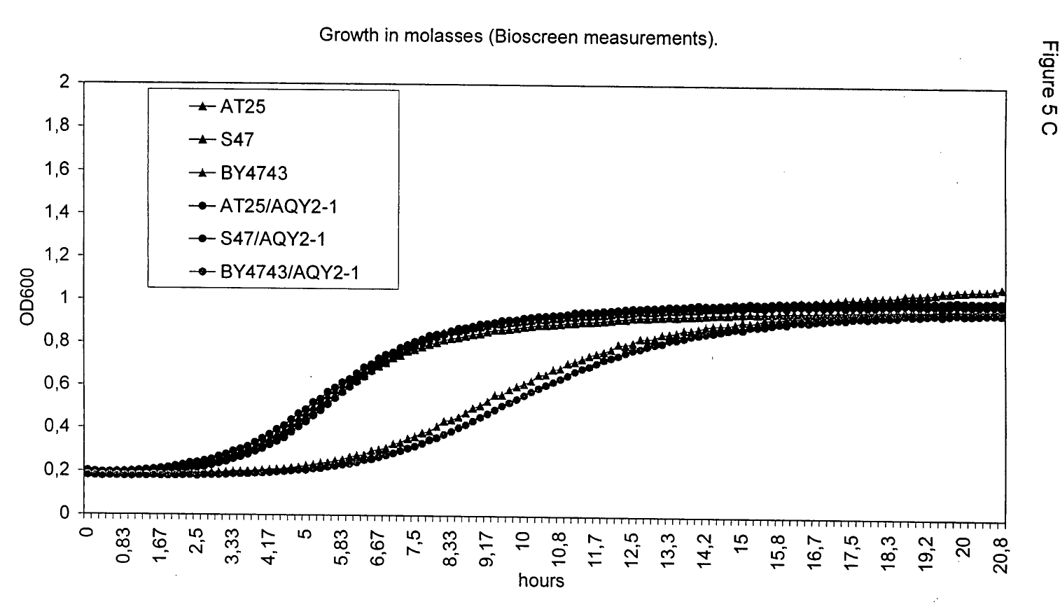

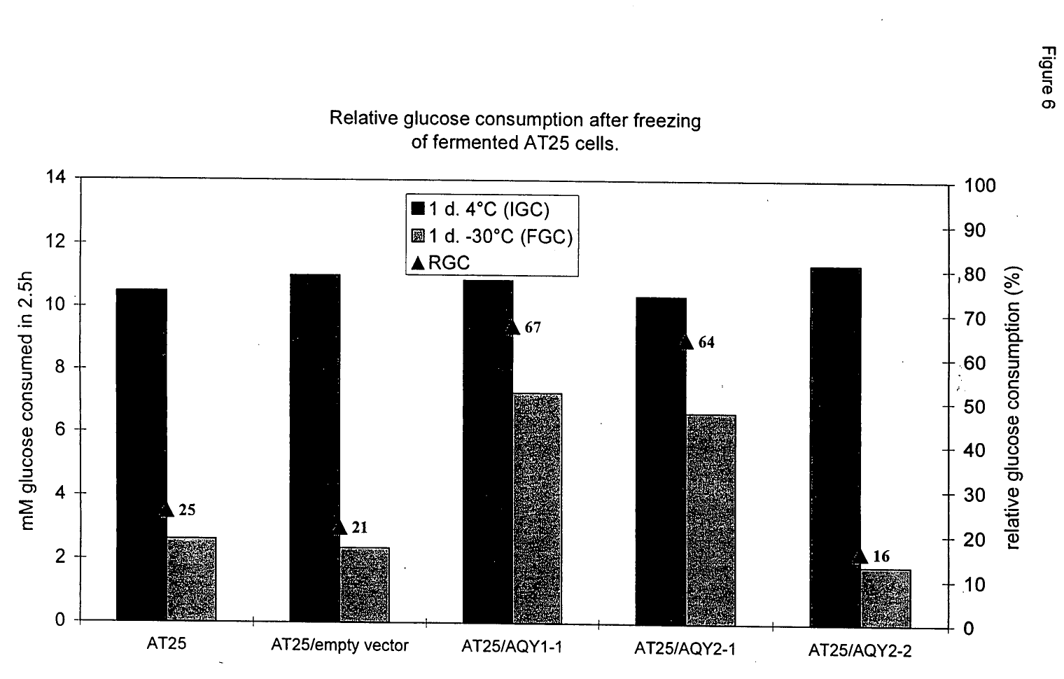

In the industrial mutant strain AT25 overexpression of AQY1-1 as well as AQY2-1 results in a drastic improvement of glucose consumption after freezing compared to 2 control strains that resp. have integrated an empty vector or a vector with the non-functional AQY2-2 allele and compared to the original AT25 strain (FIG. 6). The effect was also monitored in non-fermented cells (prior to freezing).

As shown by diagnostic restriction analysis, AT25 does not carry a functional AQY2 allele but does posses at least one functional AQY1-1 allele (FIG. 7).

Growth curves (Bioscreen measurements) did not reveal any obvious growth defect resulting from overexpression of AQY2-1-in AT25, neither for growth in YPD, nor YPM, nor molasses (FIG. 5).

Northern Analysis

To check if the improvement of freeze-resistance is correlated with higher expression levels of AQY1-1 and/or AQY2-1, Northern analysis was performed for the different laboratory and industrial backgrounds.

Results from Northern analysis of total RNA samples isolated 30 min after the resuspension of stationary phase cells in YP and 30 min after the addition of glucose in the overexpressing strains tend to show differences in mRNA stability depending on the condition and strain. In AT25, expression levels of AQY1-1 from the constitutive TPI-promotor are most pronounced 30 min after the resuspension of stationary phase cells in YP whereas overexpression of AQY2-1—is most pronounced after the addition of glucose. In the Σ1278b strain overexpressing AQY1-1 or AQY2-1, similar expression patterns are found for samples taken 30 min after resuspension of stationary phase cells in YP and for samples taken 30 min after the addition of glucose. In case of AQY1-1 overexpression, clear improvement of freeze-resistance, not only 30 min after the resuspension of stationary phase cells in YP but also 30 min after the addition of glucose, could be explained by higher protein levels of AQY1-1, as was assumed also in the case of the wild type strain. In case of AQY2-1 overexpression strain, improved freeze-resistance of fermenting cells correlates with high expression levels of AQY2-1 in this condition.

### Example 3

### Water Transport Through Aquaporins is Responsible for Improved Freeze-Resistance

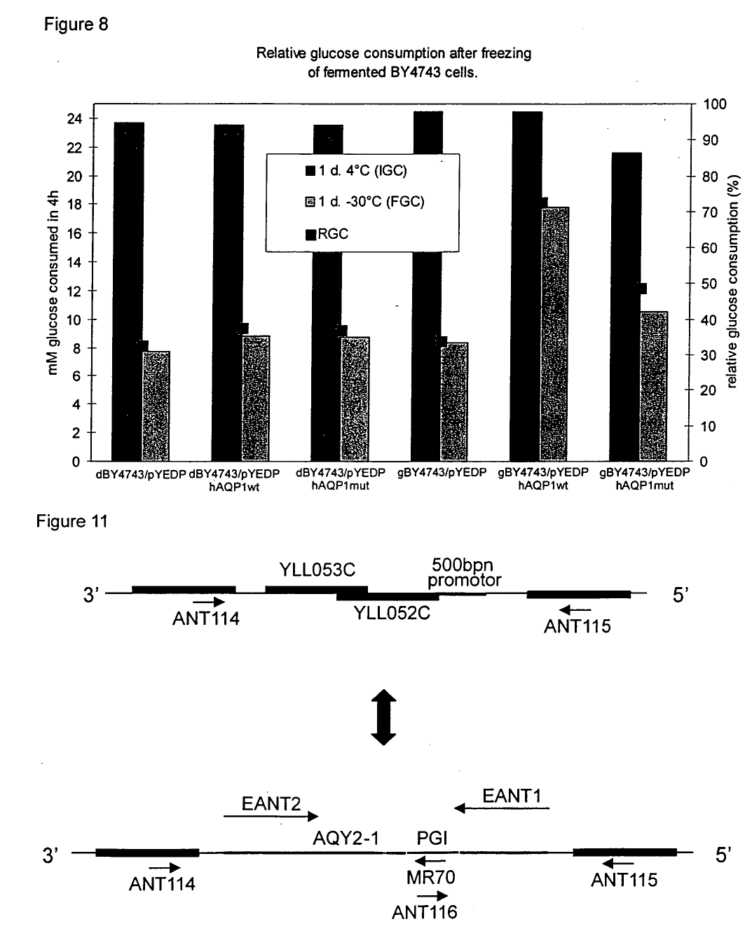

In 1995, Laizé et al. showed that the human CHIP28 water channel (hAQP1) was highly expressed, correctly localized and active upon heterologous expression in yeast. For these experiments, hAQP1 was inserted into the yeast 2μ-plasmid pYeDP-1/8-10 under the control of the inducible GAL10-CYC1 hybrid promoter and the PGK terminator, resulting in pYePD-CHIP. PYePD-CHIPmut is essentially the same construct containing a mutant hAQP1, which is expressed and localized but remains inactive. BY4743 (naturally lacking active aquaporins),was transformed with the plasmid containing the wild type hAQP1, the mutant hAQP1 and an empty plasmid. The effect on glucose consumption after freezing was tested for cells grown in YPD and YPG. When cells are grown in YPD, little or no induction of the GAL10-CYC1 promoter is expected (expression is repressed in the presence of glucose), whereas high expression levels are expected when transformants are grown in YPG. For YPD-grown cells (FIG. 8, first three double bars, prefix ‘d’), no improvement of freeze-resistance is observed in fermenting cells with the AQP1-containing plasmids, as expected. For YPG-grown cells (figure, last three double bars, prefix ‘g’), a significant improvement in glucose consumption after freezing is shown for fermenting cells bearing the construct with the wild type hAQP1, not the mutant hAQP1, compared to cells bearing the empty plasmid. In cells expressing the poorly functional allele, only a partial effect was observed.

These results clearly show the positive effect on freeze-resistance of the induction of hAQP1-expression in yeast cells. In addition, it is shown that this effect is not only due to the bare presence of the aquaporin in the membrane, since an active protein is needed.

### Example 4

### AQY1-1 and AQY2-1 Deletion Strains are More Sensitive to Freezing Compared to Wild Type Σ1278b in Distinct Conditions

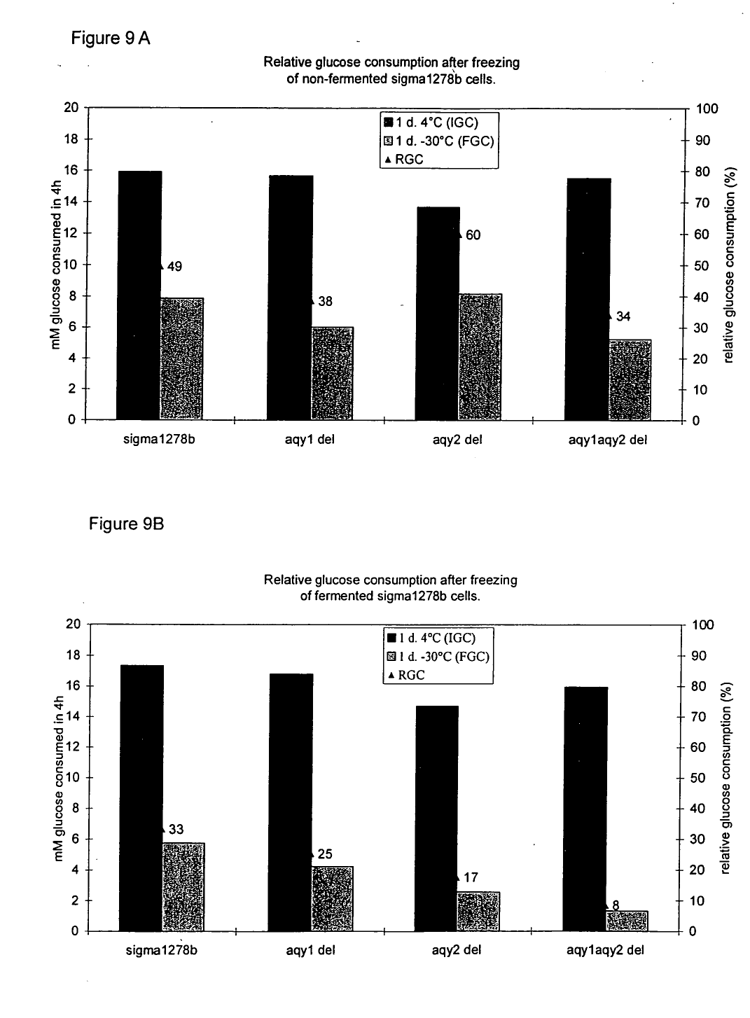

Results of glucose consumption measurements after freezing show that deletion of AQY1-1 in Σ1278b results in more freeze-sensitive cells when frozen 30 min after resuspension of stationary phase cells in YP, whereas deletion of AQY2-1 has no effect on freeze-resistance in these conditions. Both deletions seem to affect freeze-resistance of fermented cells, AQY2-1 deletion to a larger extent than AQY1-1 deletion (FIG. 9). Accordingly, results of Northern analysis show that AQY1 is induced 30 min after the resuspension of stationary phase cells in YP and repressed again 30 min after the addition of glucose, AQY2 is induced 30 min after the addition of glucose.

According to micro-array data, AQY2 seems to be expressed only in rapidly growing cells, explaining the minor effect of deletion at the onset of fermentation. In additional Northern analysis experiments we also noticed an upregulation of AQY2 in these conditions for industrial strains AT25 and S47. According to micro-array data, expression of AQY1 only was detected when cells are shifted to sporulation conditions (Chu et al., 1998) and to some extent after the diauxic shift, but these results were not confirmed at the protein level (Meyrial et al., 2001). We noticed that resuspending stationary phase cells in YP seems to induce AQY1 and deletion of this gene seems to be correlated with a loss of resistance against freezing particularly in these condition, but also 30 min after the addition of glucose. In additional Northern analysis experiments we also noticed an upregulation of AQY1 in these conditions for industrial strains AT25 and S47.

### Example 5

### The Positive Effect of AQY2-1 Overexpression is Pronounced Enough to Enable Selection of Transformed Strains Solely Using Freeze/Thaw Cycling as Selection Treatment

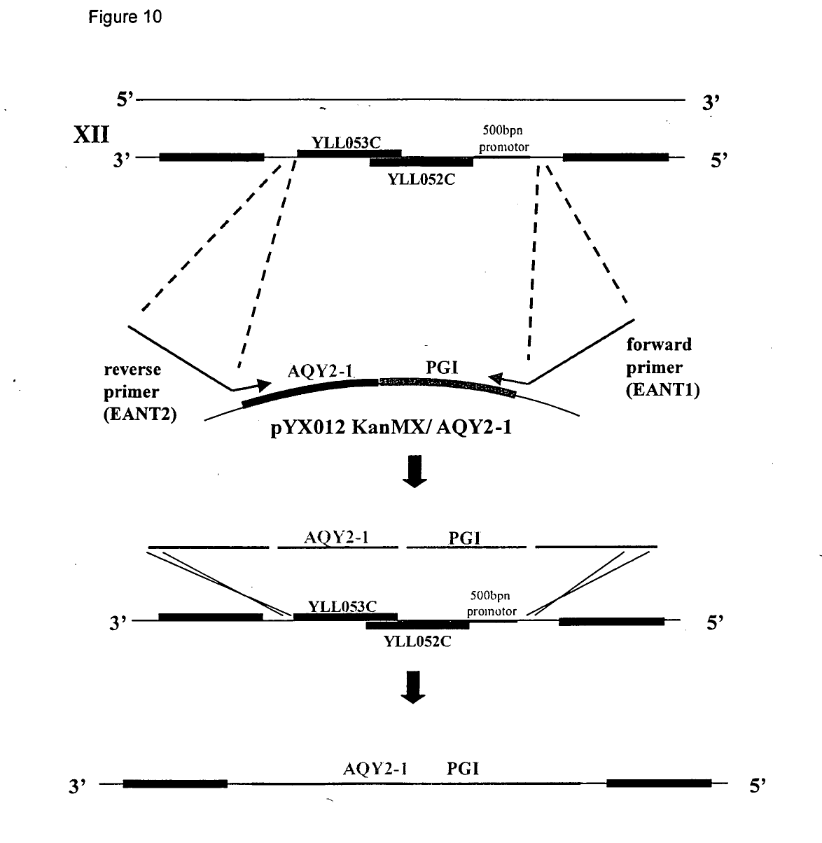

A construct is designed to replace the sequence ‘promotor/YLL052C/YLL053C’ on (at least) one of the copies of chromosome 12 in AT25 by the sequence ‘PGIpromotor/AQY2-1’ via homologuous recombination (FIG. 10). A control PCR on genomic DNA isolated from one half of the pool of transformed cells reveals that at least in some of the cells the construct is present. The construct doesn't contain a selectable marker, which implies the need for another method to select for the transformants/recombinants. On the base of the observation that the freeze-resistance (determined as glucose consumption after freezing) of AT25 having incorporated the integrative plasmid pYX012KanMX/AQY2-1 is clearly higher compared to AT25, the second half ot the transformed cell suspension is aliquoted and enriched for the desired recombinant strains by freeze/thaw cycling (30° C./−30° C./30° C. in 2 hours). After 6 cycles are finished, 10 aliquots are plated and the 20 resulting colonies are tested for integration of the exchange construct using PCR with 3 different primer pairs (FIG. 11). PCR of one of the surviving colonies results in the expected pattern of bands for 1 of the primer sets.

### Example 6

### Improvement of Freeze Tolerance as a Selection Tool for the Isolation of Aquaporin Transformants

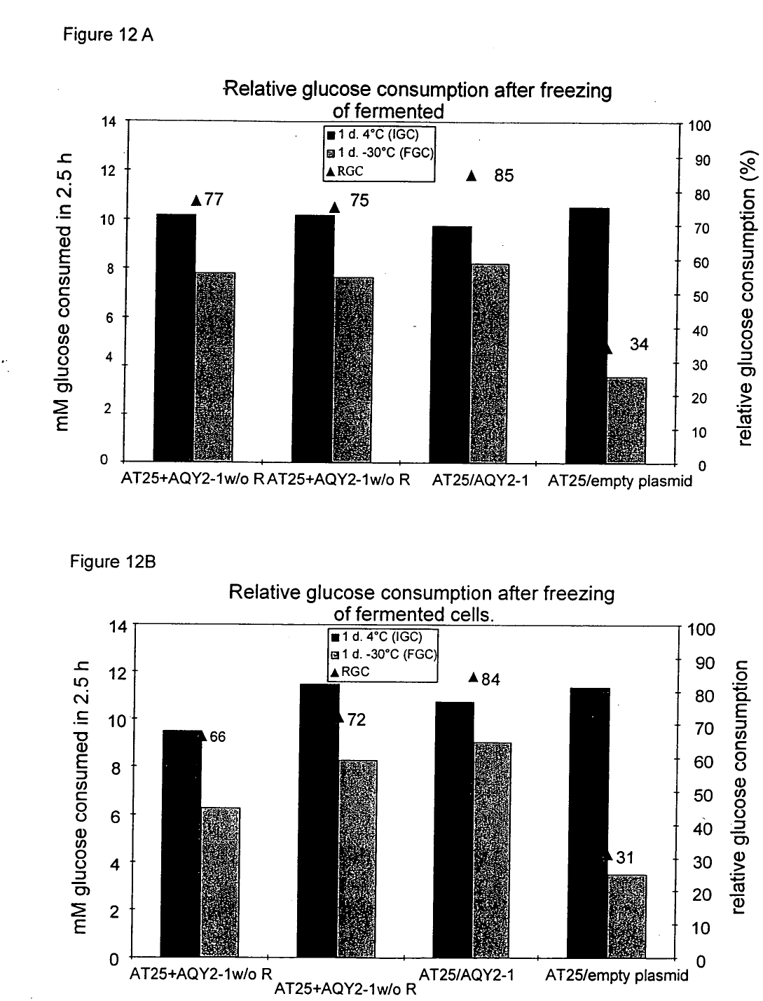

An AT25 transformant overexpressing AQY2-1 could be isolated directly on the basis of better freeze/thaw survival using six freeze/thaw cycles and PCR analysis of the surviving strains. Freeze/thaw selection on 23 aliquots each containing about 4.107 cells resulted in 23 surviving colonies (representing 2.5×10⁻⁶% survival) of which one strain contained the overexpression construct. The freeze resistance of this strain is shown in FIG. 12, and is similar to the freeze resistance of strain AT25/AQY2-1 selected directly with the use of the dominant marker. This implies that usage of an antibiotic selection marker is not required for the construction of freeze-resistant commercial yeast strains overexpressing aquaporins.

### Example 7

### The Protective Effect of AQY2-1 Overexpression During Freezing is Also Observed When Cells are Stored or Submitted to Freeze/Thaw Cycles in Frozen Dough

With both AT25 and AT25/KanMX AQY2-1 a dough was made, divided and fermented for 30 min. All small doughs were put at −30° C. in the cryostate for 1 hour except for 2 non-frozen controls. Part of the doughs was subsequently stored in the freezer (at −30° C.), part of the doughs was put in the cryostate and subjected to freeze/thaw cycles (30° C./−30° C./30° C. in 2 hours). For each measuring point (resp. 1, 10, 12, 22, 34, 46, 58, 63, 75, 83 freeze/thaw cycles and 2, 5, 12, 20, 27, 40, 50, 75, 106, 154, 195, 273 days in the freezer) the survival of yeast cells was determined in duplo. The results are summarized in Table 2 and show clearly that the aquaporin overexpressing strain survives better during frozen storage, as well as during subsequent freeze/thaw cycling.

### Example 8

### Enhanced Freeze Tolerance in Schizosaccharomyces pombe by Heterologous Overexpression of the Baker's Yeast AQY2-1 Gene

Aquaporins of both Arabidopsis thaliana and Saccharomyces cerevisiae were overexpressed in S. pombe and the effect on freeze tolerance was tested.

In the expression vector pREP HAN 41 (Craven, et al., 1998) containing the thiamine repressible NMT1-promotor and terminator and a LEU2 auxotrophic marker gene, Nicotiana tabacum aquaporin RD28 and Saccharomyces cerevisiae aquaporin AQY2-1 were cloned in frame with the HA-tag (N-terminal). The former was subcloned from pBlueScriptRD28 (Daniels, et al., 1994) using NdeI and BamHI. The latter was subcloned from pYX242/AQY2-1 (Meyrial, et al., 2001) using BamHI and filled-in EcoRI and NdeI ends. Correct cloning of RD28 and AQY2-1 in frame with the triple HA-tag was verified by sequence analysis. Transformants were selected on EMM-medium (Q-BIOgene) lacking leucine. Repressive conditions for the NMT1-promotor were created by adding thiamine to the medium to a final concentration of 5 μg/ml. It has been shown that this concentration provides sufficient repression of the NMT1-promotor (Maundrell, 1990). To test freeze tolerance of wild type 972 leu 1-32 h-cells transformed by an empty plasmid, the AQY2-1 overexpression plasmid or the RD28 overexpression plasmid, cells were pre-grown in cultures of 10 ml EMM-medium with or without thiamine for two days at 30° C. in an orbital shaker. From this pre-culture, an adequate volume was inocculated in 125 ml EMM-medium with or without thiamine to reach late exponential phase the next day. In these conditions, the repression was expected to be sufficient (Maundrell, 1990). Subsequently, equal amounts of cells (corresponding to 1 ml culture with an OD₆₀₀=20) were collected, washed and resuspended in 1 ml ice-cold 0.5% (w/v) yeast extract. Then, the cell suspensions were divided: two aliquots (40 μl each) were kept on ice and two aliquots (40 μl each) were frozen in an ethanol bath for 30 min at −30° C.

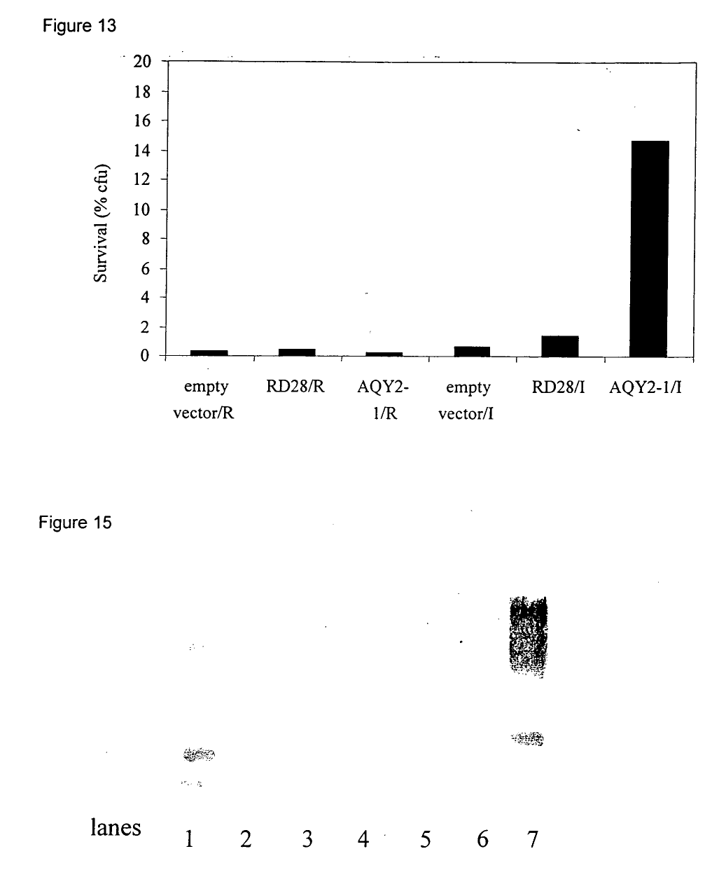

After one hour, control samples and thawed test samples were diluted in ice-cold water, plated on YE-plates and grown for 2 days at 30° C. The percentage survival was determined as the number of CFU of frozen samples compared to control samples. In general, wild type cells turned out to be very sensitive to fast freezing at −30° C. (FIG. 13). In non-repressive conditions of the NMT1-promotor, a significant improvement of freeze stress survival could be observed in cells overexpressing the S. cerevisiae aquaporin AQY2-1 gene as compared to cells containing an empty plasmid (FIG. 13). Expression of the A. thaliana aquaporin RD28 gene resulted only in a limited improvement of freeze resistance, due to the low expression of the gene. Indeed, no expression of RD28 could be detected in Northern analysis. In ‘repressive’ conditions of the NMT1-promotor, no effect was noticed, as expected (FIG. 13).

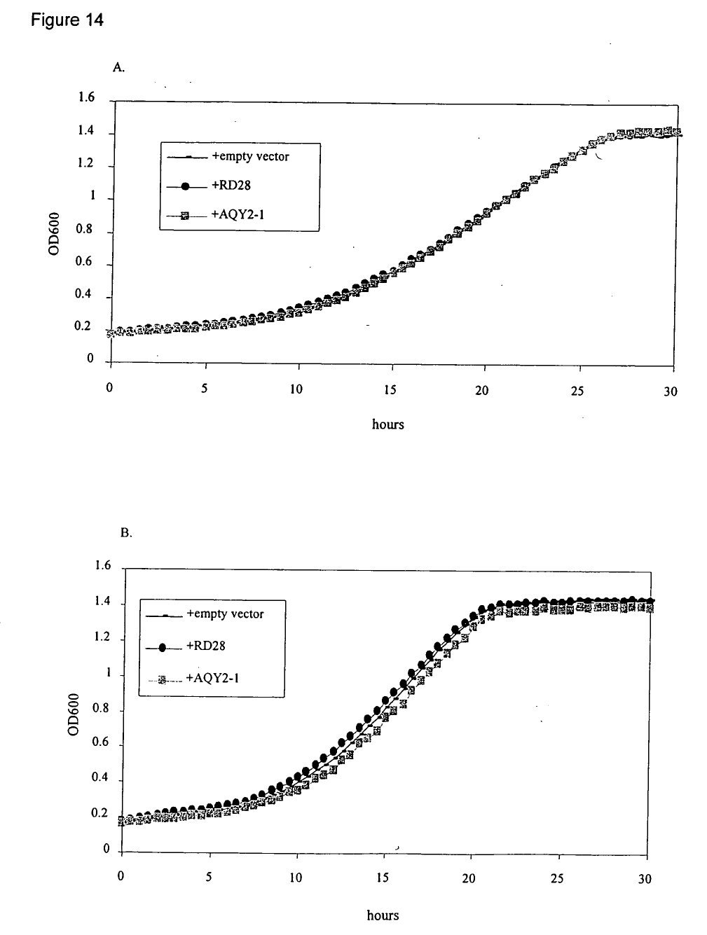

To exclude a possible effect of aquaporin expression on growth, the length of the lag phase and the maximum growth rate of the strains in EMM-medium with and without thiamine was monitored automatically at OD₆₀₀ using a BioscreenC apparatus (Labsystems). The parameters were as follows: 30° C., 250 μl culture in each well, 30 s shaking each min (medium intensity), OD₆₀₀-measurement each 30 min. Readings are saturated at OD₆₀₀-values above 1.2. No difference in growth characteristics could be monitored between the strains tested (FIG. 14).

To correlate the improved freeze resistance with aquaporin expression levels, Western analysis was performed in the same conditions. Cells were harvested and washed with ice-cold water and breaking buffer (16.1 g Na₂HPO₄.7H₂O, 5.5 g NaH₂PO₄.H₂O, 7.5 g KCl, 246 mg MgSO₄.7H₂O, pH7.0) respectively.

Subsequently, 1 ml breaking buffer, 500 μl amount of cold glass beads and 10 μl 1 mM PMSF was added to the cells. The mixture was then vortexed two times for three min at 4° C., cooling cells on ice in between. The total protein extract was centrifugated for 20 min at 4° C. and supernatant was taken. Protein concentrations were determined using the Bradford method (Biorad) with thyroglobuline as a standard. After addition of sample buffer and denaturing by boiling for 10 min, proteins (100 μg) were separated by SDS-PAGE (12.5% gel) and blotted onto nitrocellulose filters (HybondC extra, Amersham). 10 μl TriChromRanger™ (Pierce) was loaded as molecular weight marker. To confirm equal protein loads for each lane, gels were stained using 0.25% Coomassie brilliant blue in 30% MeOH, 10% acetic acid and destained in the same solution without the dye. The filters were blocked by incubation in 2% BSA in TBST (25 mM Tris/HCl pH 8,150 mM NaCl, 0.05% (v/v) Tween20) for 1 hour at room temperature. The filters were then probed with primary antibody (anti-HA high affinity Roche 1 867 423) (1:1000 dilution) overnight at room temperature in the corresponding blocking buffer. Subsequently, the filters were washed three times with TBST and incubated with alkaline phosphatase conjugated secondary antibody (anti-rat Sigma A-6066) (1:10000 dilution) in blocking buffer. Bands were detected by incubating the filters with 50 mg/ml 5-bromo-4-chloro-3-indolyl phosphate and 75 mg/ml nitroblue tetrazolium salt in alkaline phosphatase developping buffer (100 mM Tris, 100 mM NaCl, 50 mM MgCl₂ pH 8). In correlation with the freeze tolerance data, only in case of the AQY2-1 overexpression strain, a clear signal was monitored in non-repressible conditions of the NMT1-promotor (FIG. 15).

### Example 9

### Deletion of Both Alleles of the Aquaporin Encoding Gene AQY1 Significantly Reduces Freeze Tolerance of Candida albicans

Recently, a functional water channel has been described in Candida albicans (Carbrey et al., 2001b). Deletion of AQY1 resulted in a moderately decreased sensitivity to osmotic shock (Carbrey et al., 2001b), a similar but more pronounced phenotype has been reported for an aquaporin null strain of baker's yeast Saccharomyces cerevisiae (Bonhivers et al., 1998, Carbrey et al., 2001a). Freeze tolerance of heterozygous and homozygous AQY1 deletion strains were tested to check freeze tolerance in C. albicans.

The C. albicans strains described in Table 1 were grown overnight in both YPD (1% w/v yeast extract, 2% w/v bactopepton, 2% glucose) and uracil-deficient minimal medium (27 g/l dropout base, 0.77 g/l complete supplement mixture minus uracil, BIO101) at 37° C. in an orbital shaker.

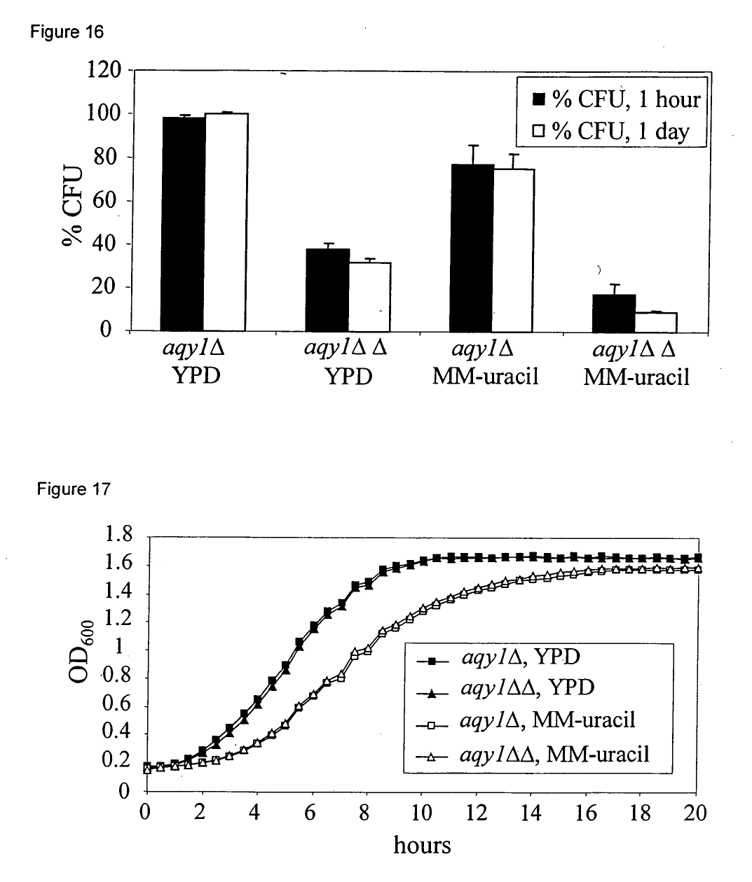

Equal amounts of cells (corresponding to 1 ml culture with an OD₆₀₀=20) were collected, washed and resuspended in 1 ml ice-cold YP. Then, cell suspensions were divided: four aliquots (40 μl each) were kept on ice and four aliquots (40 μl each) were frozen in an ethanol bath at −30° C. After both 1 hour and 1 day, two control samples and two thawed test samples were diluted in ice-cold water, plated on YPD-plates and grown for 2 days at 30° C. The percentage survival was determined as the number of CFU of frozen samples compared to control samples. Whether grown in YPD (stationary phase cells) or uracil-deficient minimal medium (exponential phase cells), the aquaporin null strain showed a significant reduction of freeze tolerance compared to the strain still carrying one AQY1-allele (FIG. 16). The latter displayed a level of freeze tolerance similar to the CAI4 URA3+ strain, indicating that the presence of one single AQY1-allele is sufficient to provide the freeze tolerance observed in this experiment.

To rule out important differences in growth characteristics between the strains tested, which by itself could influence stress resistance, the length of the lag phase and the maximum growth rate in YPD and uracil-deficient minimal medium was monitored automatically at OD₆₀₀ using a BioscreenC apparatus (Labsystems). The parameters were as follows: 37° C., 250 μl culture in each well, 30 s shaking each min at medium intensity, OD₆₀₀-measurement each 30 min. Readings are saturated at OD₆₀₀-values above one. No difference in growth characteristics could be monitored between heterozygous and homozygous AQY1 deletion strains (FIG. 17).

### Example 10

### The Improvement of Freeze Tolerance of Industrial Strain A T25 by Aquaporin Overexpression is More Pronounced in Fast Freezing Conditions

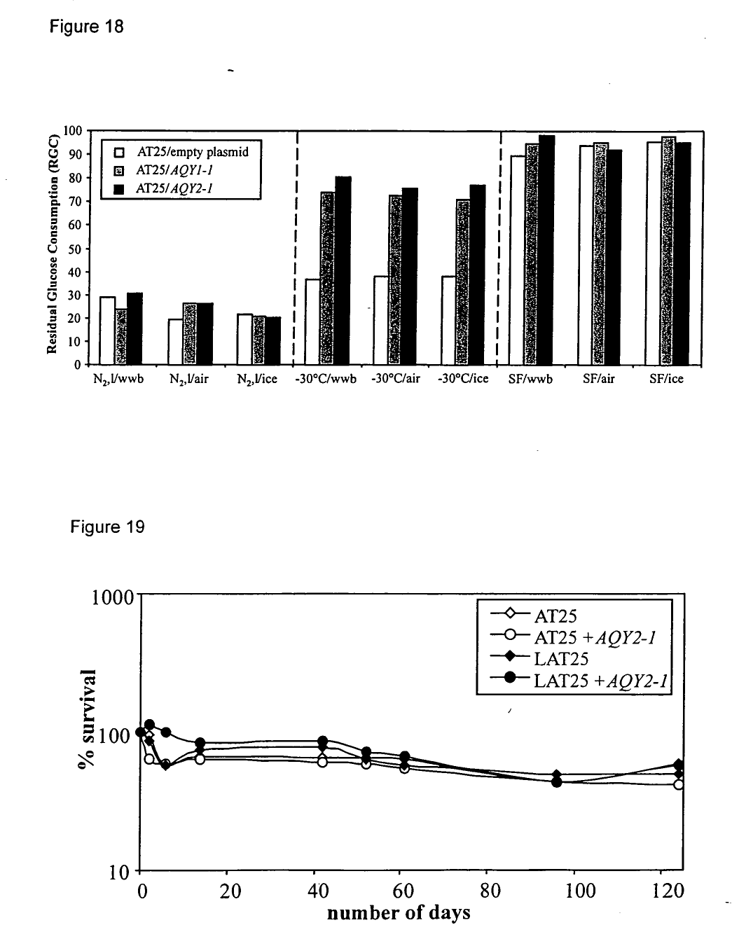

The tolerance of AT25 as well as AT25 overexpressing AQY1-1 and AQY2-1 against three different freezing conditions was tested by freezing cell suspensions in liquid nitrogen, in an ethanol bath at −30° C. and by gradual cooling at 2° C. per minute. As expected, the cells maintain a high viability during slow freezing, whereas after immersion in liquid nitrogen cells survival is dramatically decreased (FIG. 18). Aquaporin overexpression strains are significantly more freeze tolerant compared to the control strain when frozen at −30° C., as seen before. On the contrary, upon slow freezing only a small difference between the aquaporin overexpression strains and the control strain was observed. Similar results were observed in frozen doughs upon slow freezing: the presence of aquaporins has a limited advantage for the survival of yeast cells in this condition (FIG. 19). In fast freezing conditions the presence of aquaporins seems to be far more advantageous for the survival of yeast cells. In combination with each of the freezing conditions, three different thawing conditions were applied: heating in a water bath at 30° C., putting in air at room temperature and putting on ice. Only small differences in RGC were observed between the various conditions of thawing (FIG. 18).

### Example 11

### The Resistance Against Freeze-Drying of Industrial Mutant Strain AT25 is Improved Upon Aquaporin Overexpression

To be able to distinguish between the effect of freezing and drying on the glucose consumption capacity of the studied yeast cells, cells were rapidly frozen at −30° C. in an ethanol bath and after one day frozen preservation exposed to freeze-drying stress during two hours. As expected, freezing followed by freeze-drying is more detrimental to yeast cells than only freezing: after freeze-drying of AT25, the RGC was only about 20% for non-fermenting cells and about 10% for fermenting cells (Table 3), whereas after the initial freezing step, RGC-values were about 30% in both cases. In general, fermenting cells were more sensitive to freezing and freeze-drying compared to non-fermenting cells. As seen before, aquaporin overexpression in the freeze-tolerant mutant AT25 resulted in a significant further improvement of freeze tolerance. In addition, a better survival of the freeze-drying process was observed. The better survival after freeze-drying of non-fermenting cells seems mainly caused by a better survival of the freezing process, not the drying process. In case of fermenting cells, both freezing and drying processes are survived better in aquaporin overexpression strains. In accordance with results reported by other authors, no residual glucose consumption could be detected when yeast cells of strain AT25 were exposed to freeze-drying stress without prior freezing. However, for AT25 overexpressing AQY1-1 and AQY2-1, a small percentage survived.

### Example 12

### Overexpression of Aquaporin in BY2 Cells Leads to Increased Freezing Tolerance

To check if aquaporin induced freezing tolerance can also be obtained in plant cells, aquaporins of Arabidopsis thaliana and Saccharomyces cerevisiae were overexpressed in Nicotiana tabacum and the effect on freeze tolerance was tested.

Plasmids.

A. thaliana aquaporin RD28 and S. cerevisiae aquaporin AQY2-1 were cloned in the expression vector pBN35 containing a strong, constitutive 35S-promotor, a NOS-terminator and a NPTII resistance marker, resulting in plasmids pBN35/AQY2-1 and pBN35/RD28. The former was amplified from pBlueScript/RD28 (Daniels, et al., 1994) (kindly provided by Mark Daniels) using primers with BamHI and XmaI flanking sites. The latter was amplified from pYX242/AQY2-1 (Meyrial, et al., 2001) (kindly provided by Vincent Laizé) using primers with BamHI and KpnI flanking sites. Correct cloning of RD28 and AQY2-1 and the absence of PCR-introduced mutations was verified by sequence analysis.

BY-2 Transformation.

Agrobacterium tumefaciens mediated transformation of N. tabacum BY-2 cell suspensions were performed as described in Geelen, 2001. pBN35, pBN35/AQY2-1 and pBN35/RD28 transformants were selected and grown on plates of BY-2 medium (4.302 g MS salts, 0.2 g KH₂PO₄, 30 g sucrose per liter, pH 5.8) supplemented with BY-2 vitamins (0.02 g 2.4 D, 0.05 g thiamin, 5 g myo-inositol per 50 ml) and antibiotics (500 μg/ml carbenicillin, 200 μg/l vancomycin and 100 μg/ml kanamycin) at 26° C. in the dark. After 10-14 days, calli were picked and transferred to a fresh selective plate.

Cell Death Assay.

Cell death assays were essentially performed as described by Levine and co-workers (Levine, et al., 1994). Calli of considerable size were divided and separate wet weights were determined (about 10 mg). Subsequently, cells were either kept on ice or frozen in a cryostat (Haake) at −10° C. for 30 min. or at −30° C. for 15 min. Cells were then resuspended in 250 μl 0.1% Evans blue (SigmaDiagnostics) in BY-2 medium, incubated during 30 min at room temperature and washed with BY-2 medium till the supernatant remained colourless. The cell content was extracted in 1 ml 50% EtOH, 1% SDS in H₂O at 50° C. during 30 min. As measure for the amount of dead cells, the absorbance of the supernatant was measured at 600 nm.

Results

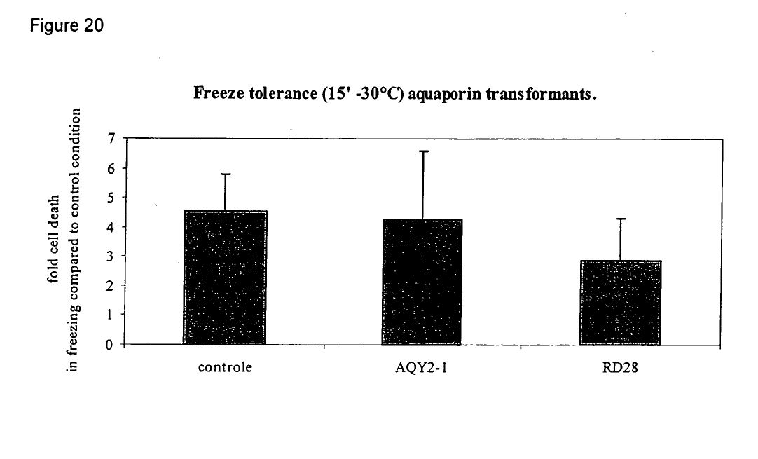

The results are summarized in FIG. 20. Both the yeast aquaporin AQY2-1 and the A. thaliana aquaporin RD28 do confer freezing tolerance to the tobacco BY2 cells. The effect of RD28 is more pronounced, but this effect is merely due to the higher expression of this gene in the BY2 cells.

TABLE 1
Yeast strains, plasmids and primers

| | | |
|---|---|---|
| Strain | genotype | source, references |

Industrial baker's yeast strains

| | | |
|---|---|---|
| S47 | polyploid, aneuploid, prototrophic | Lesaffre |
| | | Developpement |
| AT25 | polyploid, aneuploid, prototrophic | EP0967280 |
| SS1 | polyploid, aneuploid, prototrophic | |
| HAT36, HAT43, | polyploid, aneuploid, prototrophic | |
| HAT44 | | |
| ANT23 | AT25/ pYX012 KanMX AQY1-1 | this study |
| ANT1 | AT25/ pYX012 KanMX AQY2-1 | this study |
| ANT2 | AT25/ pYX012 KanMX AQY2-2 | this study |
| ANT6 | AT25/ pYX012 KanMX | this study |

Laboratory S. cerevisiae strains

| | | |
|---|---|---|
| BY4743 | MATa/alpha his3D1 leu2D0 ura3D0 | Research Genetics |
| 10560-6B | MATalpha leu2::hisG trp1::hisG | S. Hohmann |
| | his3::hisG ura3-52 | |
| YSH 1170 | MATalpha leu2::hisG trp1::hisG | S. Hohmann |
| | his3::hisG ura3-52 aqy1::kanMX4 | |
| YSH 1171 | MATalpha leu2::hisG trp1::hisG | S. Hohmann |
| | his3::hisG ura3-52 aqy2::HIS3 | |
| YSH 1172 | MATalpha leu2::hisG trp1::hisG | S. Hohmann |
| | his3::hisG ura3-52 aqy1::kanMX4 | |
| | aqy2::HIS3 | |
| ANT25 | BY4743/ pYX012 KanMX AQY1-1 | this study |
| ANT8 | BY4743/ pYX012 KanMX AQY2-1 | this study |
| ANT10 | BY4743/ pYX012 KanMX AQY2-2 | this study |
| ANT18 | BY4743/ pYX012 KanMX | this study |
| ANT27 | 10560-6B/ pYX012 KanMX AQY1-1 | this study |
| ANT26 | 10560-6B/ pYX012 KanMX AQY2-1 | this study |
| ANT28 | 10560-6B/ pYX012 KanMX AQY2-2 | this study |
| ANT29 | 10560-6B/ pYX012 KanMX | this study |
| W303-1A | MATα leu2-3, 112 ura3-1 trp1-92 his3- | Thomas and Rothstein, |
| | 11, 15 ade2-1 can1-100 GAL SUC mal | 1989 |

Schizosaccharomyces pombe

| | |
|---|---|
| 972 leu 1-32-h | L. Deveylder |

Candida

albicans

| | | |
|---|---|---|
| CAI4 | ura3Δ::imm⁴³⁴/ ura3Δ::imm⁴³⁴ | Fonzi et al, 1993 |
| CAI4 URA+ | ura3Δ::imm⁴³⁴/ ura3Δ::imm⁴³⁴ | L. De rop |
| | rp10::URA3 | |
| JC0186 (aqy1Δ) | ura3Δ::imm⁴³⁴/ ura3Δ::imm⁴³⁴ | Carbrey et al., 2001b |
| | AQY1/aqy1Δ::hisG-URA3-hisG | |
| JC0188 | ura3Δ::imm⁴³⁴/ ura3Δ::imm⁴³⁴ | Carbrey et al., 2001b |
| (aqy1ΔΔ) | aqy1Δ::hisG-URA3-hisG/aqy1Δ::hisG | |

| | | |
|---|---|---|
| Plasmid | description | source, references |

| | | |
|---|---|---|
| pUG6 | loxP-KanamycinMX-loxP cassette | Güldener et al., 1996 |
| pYX012 | integrative plasmid containing TPI | Novagen |
| | promotor and URA3 marker | |
| pYX012KanMX | pYX012 URA3::KanMX cassette | this study |
| pYX012KanMX/AQY1-1 | AQY1-1 cloned into pYX012KanMX | this study |
| pYX012KanMX/AQY2-1 | AQY2-1 cloned into pYX012KanMX | this study |
| pYX012KanMX/YLL052 | AQY2-2 cloned into pYX012KanMX | this study |
| 053C | | |
| pYX242/AQY2-1 | AQY2-1 cloned into pYX242 | Meyrial et al., 2001 |
| PYeDP1/8-10 | 2μ-plasmid containing GAL10-CYC1 | |
| | hybrid promotor and URA3 marker | |
| pYeDP-CHIP | hAQP1wt cloned into plasmid pYeDP | Laizéet al., 1995 |
| pYeDP-CHIPmut | hAQP1mut cloned into plasmid | S. Hohmann |
| | pYeDP | |
| pREP HAN 41 | | Craven, et al., 1998 |
| pBlueScript/RD28 | | Daniels, et al., 1994 |
| pCaEXP | containing URA3 and RP10 | Care et al., 1990 |

| | |
|---|---|
| Primer | Sequence |

| | | |
|---|---|---|
| AQY11-FP (ANT108) | 5′GCgaattcTTAACTATAACATGTCTTCGAA | Laizéet al., |
| | CG 3′ | 2000 |
| AQY11-RP | 5′CCGAAGCTTAAAAACACTAATTACCTCA | Laizéet al., |
| (ANT109) | GTAG 3′ | 2000 |
| AQY21-FP (ANT110) | 5′GCgaattcATGTCTAACGAATCTAACGAC | Laizéet al., |
| | C 3′ | 2000 |
| AQY21-RP | 5′CGggatccGAGCAGACTACTCTCAGTCTT | Laizéet al., |
| (ANT111) | CC 3′ | 2000 |
| AQY22-FP (ANT106) | 5′ATgaattcATGTCTAACGAATCTAACG 3′ | this study |
| AQY22-RP (ANT107) | 5′ATcccgggGTCTTCCTTCTTTTGACCTG 3′ | this study |
| TPIprom-FW | 5′ CCTACGTTAGTGTGAGCG 3′ | this study |
| TPIprom-RW | 5′ CGCTCACACTAACGTAGG 3′ | this study |
| KanFW | 5′ GGATGTATGGGCTAAATG 3′ | this study |
| KanRW | 5′ CCTCGACATCATCTGCCC 3′ | this study |
| EANT1 | 5′AGACGATGTCTAATAAATCCGGTACTT | this study |
| | CTTTACTTGCAATTAATTACTAAGCGGCG | |
| | CCTGTGTT 3′ | |
| EANT2 | 5′TAAATTAAACTACGATGGGAGCGTTAT | this study |
| | GCCAAAAAAGATAAAATTCTGAGCAGACT | |
| | ACTCTC 3′ | |
| ANT114 | 5′ GCGGACATCGATGCAGG 3′ | this study |
| ANT115 | 5′ GGAAAGAATGGATAGTGGTA 3′ | this study |

Survival in dough
A. Freeze/thaw cycling

| | Number of cycles | | | | | | | | | | |
|---|---|---|---|---|---|---|---|---|---|---|---|
| | 0 | 1 | 10 | 12 | 22 | 34 | 46 | 58 | 63 | 75 | 83 |
| AT25 | 100 | 52 | 43 | 37 | 31 | 30 | 24 | 13 | 12 | 5 | 5 |
| AT25 + AQY2-1 | 100 | 91 | 159 | 121 | 110 | 33 | 42 | 44 | 18 | 5 | 6 |

B. Frozen storage

| | Number of days | | | | | | | | | | | | |
|---|---|---|---|---|---|---|---|---|---|---|---|---|---|
| | 0 | 2 | 5 | 12 | 20 | 27 | 40 | 50 | 75 | 106 | 154 | 195 | 273 |
| AT25 | 100 | 61 | 49 | 43 | 42 | 38 | 47 | 30 | 12 | 9 | 5 | 2 | 2 |
| AT25 + AQY2-1 | 100 | 92 | 63 | 81 | 61 | 55 | 82 | 67 | 57 | 50 | 55 | 15 | 15 |

TABLE 3
Residual glucose consumption (RGC) of frozen cells
compared to non-frozen (NF) cells (left), RGC of
frozen/freeze-dried (FD) cells compared to non-frozen cells
(right) and RGC of frozen/freeze-dried cells compared to
frozen cells (middle).

| | | | RGC, |
|---|---|---|---|
| | RGC, | RGC, −30° C. + | −30° C. + |
| | −30° C. vs NF | FD vs −30° C. | FD vs NF |
| non-fermenting cells | | | |
| AT25/empty plasmid | 32.5 | 60.0 | 19.5 |
| AT25/AQY2-1 | 100.0 | 65.8 | 66.0 |
| AT25/AQY1-1 | 74.0 | 64.4 | 47.6 |
| fermenting cells | | | |
| AT25/empty plasmid | 27.7 | 37.2 | 10.3 |
| AT25/AQY2-1 | 84.7 | 75.7 | 64.1 |
| AT25/AQY1-1 | 65.3 | 73.2 | 47.9 |

### References

Almeida, M. J. and Pais, C. (1996). Leaving ability and freeze tolerance of yeasts isolated from traditional corn and rye bread doughs. Appl Environ Microbiol, 62, 4401-4404.

Attfield, P. V. (1997). Stress tolerance: the key to effective strains of industrial baker's yeast. Nat Biotechnol, 15, 1351-1357.

Attfield, P. V., Raman, A. and Northcott, C. J. (1992). Construction of Saccharomyces cerevisiae strains that accumulate relatively low concentrations of trehalose, and their application in testing the contribution of the dissaccharide to stress tolerance. FEMS Microbiol Lett, 73, 271-276.

Balk, P. A. and de Boer, A. D. (1999). Rapid stalk elongation in tulip (Tulipa gesneriana L. cv. Apeldoorn) and the combined action of cold-induced invertase and the water-channel protein gammaTIP. Planta, 209, 346-354.

Bolduc, R., Comeau, A., Couture, L., Hope, H. J. and St. Pierre, C. A. (1985). Une méthode de sélection des céréales pour la resistance à I'hiver. Phytoprotection, 66, 47-52.

Bonhivers, M., Carbreys, J. M., Gould, S. J. and Agre, P. (1998). Aquaporins in Saccharomyces. Genetic and functional distinction between laboratory and wild type strains. J. Biol. Chem. 273, 27565-27572.

Carbrey, J. M., Bonhivers, M., Boecke, J. D. and Agre, P. (2001a). Aquaporins in Saccharomyces: characterization of a second functional water channel protein. Proc Natl Acad Sci USA, 98, 1000-1005.

Carbrey, J. M., Cormack, B. P. and Agre, P. (2001b). Aquaporin in Candida: characterization of a functional water channel protein. Yeast. 18:1391-1396.

Care, R. S., Trevethick, J., Binley, K. M. and Sudbery, P. E. 1990. The MET3 promotor: a new tool for Candida albicans molecular genetics. Molecular Microbiology. 34(4):792-798.

Chaumont, F., Barrieu, F., Herman, E. M. and Chrispeels, M. J. (1998). Characterization of a maize tonoplast aquaporin expressed in zones of cell division and elongation. Plant Physiol, 117,1143-1152.

Chu, S., DeRisi, J., Eisen, M., Mulholland, J., Botstein, D., Brown, P. O. and Herskowitz, I. (1998). The transcriptional program of sporulation in budding yeast. Science, 282, 1421.

Craven, R. A., Griffiths, D. J. F., Sheldrick, K. S., Randall, R. E., Hagan, I. M. and Carr, A. M. (1998). Vectors for the expression of tagged proteins in Schizosaccharomyces pombe. Gene. 221:59-68.

Daniels, M. J., Mirkov, T. E. and Chrispeels, M. J. (1994). The plasma membrane of Arabidopsis thaliana contains a mercury-insensitive aquaporin that is a homolog of the tonoplast water channel protein TIP. Plant Physiol. 106:1325-1333.

Diniz-Mendes, L., Bernardes, E., de Araujo, P. S., Panek, A. D. and Paschaolin, V. M. (1999). Preservation of frozen yeast cells by trehalose. Biotechnol Bioeng, 65, 572-578.

Fischbarg, J. Kuang, K. Y., Vera, J. C., Arant, S., Silverstein, S. C., Loike, J. and Rosen, O. M. (1990). Glucose transporters serve as water channels. Proc Natl Acad Sci USA 87, 3244-3247.

Fischbarg, J. and Vera, J. C. (1995). Multifunctional transporte models: lessons from the transport of water, sugars and ring compounds by GLUTs. Am J Physiol 268, C1077-1089.

Fonzi, W. A., and Irwin, M. Y. (1993). Isogenic strain construction and gene mapping in Candida albicans. Genetics. 134:717-728.

Gélinas P., Fiset G., LeDuy A. and Goulet J. (1989). Effect of growth conditions and trehalose content on cryotolerance of baker's yeast in frozen doughs. Appl Environ Microbiol, 55,2453-2459.

Geelen, D. N. V. and Inzé, D. G. (2001). A bright future for the Bright Yellow-2 cell culture. Plant Physiol 127, 1375-1379.

Guldener U, Heck S, Fielder T, Beinhauer J, Hegemann J H. (1996) A new efficient gene disruption cassette for repeated use in budding yeast. Nucleic Acids Res 24(13):2519-24

Hahn Y. S. and Kawai H. (1990). Isolation and characterization of freeze-tolerant yeasts from nature available for the frozen-dough method. Agr and Biol chem, 54, 829-831.

Hasegawa, H. Skach, W., Baker, O., Calayag, M. C., Lingappa, V. and Verkman, A. S. (1992). A multifunctional water channel formed by CFTR. Science 258, 1477-1479.

Hatuno, S., Udou, M., Koga, N., Honjoh, K. and Miyamoto, T. (1996). Impairment of the glycolytic system and actin in baker's yeast during frozen storage. Biosci Biotechnol Biochem, 60, 61-64.

Hino A., Takano. H. and Tanaka Y. (1987). New freeze-tolerant yeast for frozen dough preparations. Cereal Chem, 64, 269-275.

Hino A., Mihara K., Nakashima K. and Takano H. (1990). Trehalose levels and survival ratio of freeze-tolerant versus freeze-sensitive yeasts. Appl Environ Microbiol, 56,1386-1391.

Iwahashi, H., Obuchi, K., Fujii, S. and Komatsu, Y. (1995). The correlative evidence suggesting that treahlose stabilizes membrane structure in the yeast Saccharomyces cerevisiae. Cell Mol Biol, 41, 763-769.

Johansson, I., Karlsson, M., Shukla, V. K., Chrispeels, M. J., Larsson, C. and Kjellbom, P. (1998). Water transport activity of the plasma membrane aquaporin PM28A is regulated by phosphorylation. Plant Cell, 10, 451-459.

Johansson, I., Karlsson, M., Johanson, U., Larsson, C. and Kjellbom, P. (2000). The role of aquaporins in cellular and whole plant water balance. Biochim Biophys Acta, 1465, 324-342.

Kaul, S. C., Obuchi, K., Iwahashi, H. and Komatsu, Y. (1992). Cryoprotection provided by heat shock treatment in Saccharomyces cerevisiae. Cell Mol Biol, 38,135-143.

Kim, J., Alizadeh, P., Harding, T., Hefner-Gravink, A. and Klionsky, D. J. (1996). Disruption after yeast ATH1 gene confers better survival after dehydration, freezing, and ethanol shock: potential commercial applications. Appl Environ Microbiol, 62, 1563-1569.

Kline L. and Sugihara T. F. (1968). Frozen bread doughs. I-prepared by the straight dough method. II-Prepared by the sponge and dough method. Bakers Dig, 34, 44-51.

Komatsu, Y., Kaul, S. C., Iwahashi, H. and Obuchi, K. (1990). Do heat shock prteins provide protection against freezing? FEMS Microbiol Lett, 60, 159-162.

Laizé, V., Rousselet, G., Verbavatz, J. M., Berthonaud, V., Gobin, R., Roudier, N., Abrami, L., Ripoche, P. and Tacnet, F. (1995). Functional expression of the human CHIP28 water channel in a yeast secretory mutant. FEBS Lett, 373, 269-274.

Laizé V, Gobin R, Rousselet G, Badier C, Hohmann S, Ripoche P, Tacnet F. (1999). Molecular and functional study of AQY1 from Saccharomyces cerevisiae: role of the C-terminal domain. Biochem Biophys Res Commun, 257(1), 139-44

Laizé, V., Tacnet, F., Ripoche, P. and Hohmann, S. (2000). Polymorphism of Saccharomyces cerevisiae aquaporins. Yeast, 16, 897-903.

Levine, A., Tenhaken, R., Dixon, R. and Lamb, C. (1994). H2O2 from the oxidative burst orchestrates the plant hypersensitive disease resistance response. Cell, 79, 583-593

Lewis, J. G., Learmonth, R. P. and Watson, K. (1993). Role of growth phase and ethanol in freeze-thaw stress resistance of Saccharomyces cerevisiae. Appl environ Microbiol, 59,1065-1071.

Lewis, J. G., Learmonth, R. P., Attfield, P. V. and Watson, K. (1997). Stress co-tolerance and trehalose content in baking strains of Saccharomyces cerevisiae. J Ind Microbiol Biotechnol, 18, 30-36.

Li, L. G., Li, S. F., Tao, Y and Kitagawa, Y. (2000). Molecular cloning of a novel water channel from rice: its products expression in Xenopus oocytes and involvement in chilling tolerance. Plant Sci, 154, 43-51.

Loike, J. D., Cao, L., Kuang, K., Vera, J. C., Silverstein, S. C. and Fischbarg, J. (1993).Role of facilitative glucose transporters in diffusional water permeability through J774 cells. J Gen Physiol 102, 897-906.

Loike, J. D., Hickman, S., Kuang, K. Xu, M., Cao, L. Vera, J. C., Silverstein, S. C. and Fischbarg, J. (1996). Sodium-glucose transporters display sodium- and phlorizin-dependent water permeability. Am J Physiol, 271, C1774-1779.

Matsumura, Y., Uchida, S., Rai, T., Sasaki, S. and Marumo, F. (1997). Transcriptional regulation of aquaporin-2 water channel gene by cAMP. J Am Soc Nephrol, 8, 861-867.

Maundrell, K. (1990). Nmt1 of fission yeast, a highly transcribed gene completely repressed by thiamine. J. of Biol. Chem. 265(19):10857-10864.

Maurel, C. Kado, R. T., Guern, J. and Chrispeels, M. J. (1995). Phosphorylation regulates the waterchannel activity of the seed-specific aquaporin α-TIP. EMBO J, 14, 3028-3035.

Maurel, C. (1997). Aquaporins and water permeability of plants. Ann Rev Plant Physiol Plant Mol Biol, 48, 399-429.

Merrit, P. P. (1960). The effect of preparation on the stability and performance of frozen, unbaked, yeast-leavened dough. Bakers Dig, 34, 57.

Meyrial, V., Laizé, V., Gobin, R., Ripoche, P., Hohmann, S. and Tacnet, F. (2001). Existence of a tightly regulated channel in Saccharomyces cerevisiae. Eur. J. Biochem. 268, 334-343.

Murakami, Y., Yokoigawa, K., Kawai, F. and Kawai, H. (1996). Lipid composition of commercial baker's yeast having different freeze-tolerance in frozen dough. Biosci Biotechnol Biochem, 60, 1874-1876.

Nakagawa, S. and Ouchi, K. (1994). Construction from a single parent of baker's yeast strains with high freeze tolerance and fermentative activity in both lean and sweet doughs. Appl Environ Microbiol, 60, 3499-3502.

Neyreneuf, O. and Van Der Plaat, J. B. (1991). Preparation of frozen french bread dough with improved stability. Cereal Chem, 68, 60-66.

Oda, Y., Uno, K. and Ohta, S. (1986). Selection of yeasts for breadmaking by the frozen-dough method. Appl Environ Microbiol, 52, 941-943.

Park, J. I., Grant, C. M., Attfield, P. V. and Dawes, I. W. (1997). The freeze-thaw stress response of the yeast Saccharomyces cerevisiae is growth phase specific and is controlled by nutritional state via the RAS-cyclic AMP signal transduction pathway. Appl Environ Mircrobiol, 63, 3818-3824.

Park, J. I., Grant, C. M., Davies, M. J. and Dawes, I. W. (1998). The cytoplasmic Cu,Zn superoxide dismutase of Saccharomyces cerevisiae is required for resistance to freeze-thaw stress. Generation of free radicals during freezing and thawing. J Biol Chem, 273, 22921-22928.

Randez-Gil, F., Sanz, P. and Prieto, J. A. (1999). Engineering baker's yeast: room for improvement. Trends Biotechnol, 17, 237-244.

Robertson, L. S., Causton, H. C., Young, R. A. and Fink, G. R. (2000). Proc Natl Acad Sci USA, 97, 5984-5988.

Rep, M., M. Krantz, J. M. Thevelein, and S. Hohmann. 2000. The transcriptional response of Saccharomyces cerevisiae to osmotic shock—Hot1p and Msn2p/Msn4p are required for the induction of subsets of high osmolarity glycerol pathway-dependent genes. J. Biol. Chem. 275:8290-8300

Shima, J., Hino, A., Yamada-Iyo, C., Suzuki, Y., Nakajima, R., Watanabe, H., Mori, K. and Tanako, H. (1999). Stress tolerance in doughs of Saccharomyces cerevisiae trehalase mutants derived from commercial baker's yeast. Appl Environ Microbiol, 65, 2841-2846.

Tanghe, A., Teunissen, A., Van Dijck, P., Thevelein, J. M. (2000). Identification of genes responsible for improved cryoresistance in fermenting yeast cells. Int J Food Microbiol, 55(1-3), 259-62.

Takagi, H., Iwamoto, F. and Nakamori, S. (1997). Isolation of freeze-tolerant laboratory strains of Saccharomyces cerevisiae from proline-analogue-resistant mutants. Appl Microbiol Biotechnol, 47, 405-411.

Thevelein, J. M. (1994) Signal transduction in yeast. Yeast, 10, 1753-1790.

Thevelein, J. M. and de Winde, J. H. (1999) Novel sensing mechanisms and targets for the cAMP-protein kinase A pathway in the yeast Saccharomyces cerevisiae. Mol Microbiol, 33, 904-918.

Thomas B J, Rothstein R: The genetic control of direct repeat recombination in Saccharomyces: the effect of rad52 and rad1 on mitotic recombination at GAL10, a transcriptionally regulated gene. Genetics 1989,123:725-738.

Tyerman, S. D., Bohnert, H. J., Maurel, C., Steudle, E. and Smith, J. A. C. (1999) Plant aquaporins: their molecular biology, biophysics and significance for plant water relations. J. Experim. Botany, 50,1055-1071.

Van Dijck, P. Colavizza, D., Smet, P and Thevelein, J. M. (1995) Differential importance of trehalose in stress resistance in fermenting and non-fermenting Saccharomyces cerevisiae cells. Appl Environ Microbiol, 61,109-115.

Van Dijck, P., Gorwa, M. F., Lemaire, K., Teunissen, A., Versele, M., Colombo, S., Dumortier, F., Ma, P., Tanghe, A., Loiez, A. and Thevelein, J. M. (2000) Characterization of a new set of mutants deficient in fermentation-induced loss of stress resistance for use in frozen dough applications. Int J Food Microbiol, 55, 187-92.

1. The use a of protein facilitating water diffusion or water transport through the cell membrane to obtain chilling and/or freeze-tolerance in a eukaryotic cell.

2. The use of a protein according to claim 1, whereby said protein is an aquaporin or an aquaporin-like protein.

3. The use according to claim 2, whereby said aquaporin or aquaporin-like protein comprises SEQ ID N° 2.

4. The use according to claim 2, whereby said aquaporin or aquaporin-like protein comprises SEQ ID N° 4.

5. The use according to claim 2, whereby said aquaporin or aquaporin-like protein comprises SEQ ID N° 6.

6. The use according to any of the claims 1 to 5 whereby said eukaryotic cell is a yeast cell.

7. The use according to any of the claims 1 to 5 whereby said eukaryotic cell is a plant cell.

8. A method to obtain chilling and/or freeze tolerance in a eukaryotic cell, comprising a) placing a gene encoding a protein facilitating water diffusion or water transport through the cell membrane downstream a promoter sequence suitable for expressing said gene in said eukaryotic cell, b) transforming or transfecting the nucleic acid comprising said promoter and gene into said eukaryotic cell and c) growing said eukaryotic cells under conditions suitable for the expression of said gene.

9. A method to obtain chilling and/or freeze-tolerance in a eukaryotic cell, comprising the insertion of a non-endogenous promoter upstream a gene encoding a protein facilitating water diffusion or water transport through the cell membrane.

10. The method according to claim 8 or 9, whereby said protein is an aquaporin or an aquaporin-like protein

11. The method according to claim 10, whereby said gene comprises SEQ ID N° 1.

12. The method according to claim 10, whereby said gene comprises SEQ ID N° 3.

13. The method according to claim 10, whereby said gene comprises SEQ ID N° 5.

14. The method according to any of the claims 8 to 13, whereby said eukaryotic cell is a yeast cell.

15. The method according to any of the claims 8 to 13, whereby said eukaryotic cell is a plant cell.

16. The use of a compound, which activates a protein facilitating water diffusion or water transport through the cell membrane to obtain chilling and/or freeze-tolerance in a eukaryotic cell.

17. The use according to claim 16, whereby said protein is an aquaporin or an aquaporin-like protein.

18. The use according to claim 16 or 17, whereby said compound is a protein kinase.

19. The use according to claim 16 or 17, whereby said compound is an inhibitor of a phosphatase.

20. A chilling and/or freeze-tolerant eukaryotic cell, characterized by an enhanced content of a protein facilitating water diffusion or water transport through the cell membrane.

21. A chilling and/or freeze-tolerant eukaryotic cell according to claim 20, whereby said protein is an aquaporin or an aquaporin-like protein.

22. A chilling and/or freeze-tolerant eukaryotic cell according to claim 20 or 21, obtainable by a method according to any of the claims 8-15.

23. A chilling and/or freeze-tolerant eukaryotic cell according to claim 20 or 21, obtainable by the use of a compound according to any of the claims 16-19.

24. A chilling and/or freeze-tolerant eukaryotic cell according to any of the claims 20-23, whereby said eukaryotic cell is a yeast cell.

25. A chilling and/or freeze-tolerant eukaryotic cell according to any of the claims 20-23, whereby said eukaryotic cell is a plant cell.

26. A chilling and/or freeze-tolerant yeast cell, according to claim 24, whereby said yeast is baker's yeast.

27. The use of a chilling and/or freeze-tolerant baker's yeast according to claim 26 to prepare frozen dough.

28. A dough, comprising at least one yeast cell according to claim 26.

29. A plant, comprising at least one plant cell according to claim 25.


SEQUENCE LISTING (20 sequences)

SEQ ID NO: 1 - DNA, 984 bp, Saccharomyces cerevisiae
  CDS (1)..(984)
atgtcttcgaacgattcgaacgataccgacaagcaacatacacgtctggatcctaccggtgtggacgacgcctacatccctccggagcagccggaaacaaagcaccatcgctttaaaatctctagggacaccctgagaaaccactttatcgctgcggtcggtgagttctgcggcacattcatgtttttatggtgcgcttacgttatctgcaatgtcgctaaccatgatgtcgcactcgttgcagcgcctgacggttcccatccgggtcaattgattatgattgccatcggtttcggattttccgtcatgttttctatctggtgttttgccggtgtctctggtggggctttgaatcctgctgtgtcgctttcgctgtgcttggcgagagccgtctctcctacaagatgtgtcgttatgtgggtttcgcagattgttgccggaatggccgctggaggcgctgcaagcgccatgacacctggtgaagtcctctttgccaattctttgggcctgggctgctctaggacgaggggtttgttcctggagatgttcggcaccgctatcctatgtttaacagtcttaatgacggctgtggagaagcgtgaaaccaactttatggccgcgctgcccatcggcatctccctgtttatcgcacacgtcgctttgactgcatacacaggcacaggtgtcaaccctgcgaggtccttgggtgctgctgtcgcagccagatacttccctcattaccactggatttattggattggcccgctgttaggatccattttagcatggtctgtatggcaattattgcaaatcttagactacacaacctacgttaccgctgaaaaggctgccagcaccaaggaaaaagctcaaaaaaaggtgaaaccagcagttcctctgctgtggctgaagtctaattttcccctccttttctttatttctcgctcactagcacttaatgttataatattcggcaaaaactag

SEQ ID NO: 2 - protein, 327 aa, Saccharomyces cerevisiae
MSSNDSNDTDKQHTRLDPTGVDDAYIPPEQPETKHHRFKISRDTLRNHFIAAVGEFCGTFMFLWCAYVICNVANHDVALVAAPDGSHPGQLIMIAIGFGFSVMFSIWCFAGVSGGALNPAVSLSLCLARAVSPTRCVVMWVSQIVAGMAAGGAASAMTPGEVLFANSLGLGCSRTRGLFLEMFGTAILCLTVLMTAVEKRETNFMAALPIGISLFIAHVALTAYTGTGVNPARSLGAAVAARYFPHYHWIYWIGPLLGSILAWSVWQLLQILDYTTYVTAEKAASTKEKAQKKVKPAVPLLWLKSNFPLLFFISRSLALNVIIFGKN

SEQ ID NO: 3 - DNA, 870 bp, Saccharomyces cerevisiae
  CDS (1)..(870)
atgtctaacgaatctaacgaccttgaaaaaaacatttcgcacttggacccaaccggtgttgacaatgcttatattccacctgaacagccggaaacgaagcattcgcgttttaatattgacagagataccttaagaaaccactttatcgctgctgtgggtgagttttgcggtaccttcatgtttttatggtgtgcttacgtcatttgtaatgtcgctaaccatgatgtggctttgacaaccgagcctgagggctctcatccaggtcaattgatcatgattgcccttggtttcggtttctctgtgatgttttctatctggtgttttgctggtgtttctggtggggctttgaacccagccgtttctctctctttgtgtttggccagagccatctcaccagccagatgtgtagtgatgtggtttcctcagatcattgctgggatggctgctggtggtgccgctagtgctatgactccaggcaaggttctctttactaatgctttgggtttaggctgttccaggtctagggggttgtttttggaaatgtttggtactgctgtgttgtgtttaacagttttgatgactgctgttgaaaaacgtgaaactaactttatggctgcgcttccaattggtatttctttattcatggctcacatggctttgaccggttacactggtaccggtgtcaaccctgcaaggtctctaggtgccgccgttgctgccagatatttccctcattaccactggatttactggattggcccacttttgggtgccttcttagcctggtcagtgtggcaattattacaaatccttgattacactacatacgttaatgccgaaaaggcggcaggtcaaaagaaggaagactga

SEQ ID NO: 4 - protein, 289 aa, Saccharomyces cerevisiae
MSNESNDLEKNISHLDPTGVDNAYIPPEQPETKHSRFNIDRDTLRNHFIAAVGEFCGTFMFLWCAYVICNVANHDVALTTEPEGSHPGQLIMIALGFGFSVMFSIWCFAGVSGGALNPAVSLSLCLARAISPARCVVMWFPQIIAGMAAGGAASAMTPGKVLFTNALGLGCSRSRGLFLEMFGTAVLCLTVLMTAVEKRETNFMAALPIGISLFMAHMALTGYTGTGVNPARSLGAAVAARYFPHYHWIYWIGPLLGAFLAWSVWQLLQILDYTTYVNAEKAAGQKKED

SEQ ID NO: 5 - DNA, 1663 bp, Homo sapiens
  CDS (39)..(848)
gcacccggcagcggtctcaggccaagccccctgccagcatggccagcgagttcaagaagaagctcttctggagggcagtggtggccgagttcctggccacgaccctctttgtcttcatcagcatcggttctgccctgggcttcaaatacccggtggggaacaaccagacggcggtccaggacaacgtgaaggtgtcgctggccttcgggctgagcatcgccacgctggcgcagagtgtgggccacatcagcggcgcccacctcaacccggctgtcacactggggctgctgctcagctgccagatcagcatcttccgtgccctcatgtacatcatcgcccagtgcgtgggggccatcgtcgccaccgccatcctctcaggcatcacctcctccctgactgggaactcgcttggccgcaatgacctggctgatggtgtgaactcgggccagggcctgggcatcgagatcatcgggaccctccagctggtgctatgcgtgctggctactaccgaccggaggcgccgtgaccttggtggctcagccccccttgccatcggcctctctgtagcccttggacacctcctggctattgactacactggctgtgggattaaccctgctcggtcctttggctccgcggtgatcacacacaacttcagcaaccactggattttctgggtggggccattcatcgggggagccctggctgtactcatctacgacttcatcctggccccacgcagcagtgacctcacagaccgcgtgaaggtgtggaccagcggccaggtggaggagtatgacctggatgccgacgacatcaactccagggtggagatgaagcccaaatagaaggggtctggcccgggcatccacgtagggggcaggggcaggggcgggcggagggaggggaggggtgaaatccatactgtagacactctgacaagctggccaaagtcacttccccaagatctgccagacctgcatggtcaagcctcttatgggggtgtttctatctctttctttctctttctgtttcctggcctcagagcttcctggggaccaagatttaccaattcacccactcccttgaagttgtggaggaggtgaaagaaagggacccacctgctagtcgcccctcagagcatgatgggaggtgtgccagaaagtcccccctcgccccaaagttgctcaccgactcacctgcgcaagtgcctgggattctaccgtaattgctttgtgcctttgggcacggccctccttcttttcctaacatgcaccttgctcccaatggtgcttggagggggaagagatcccaggaggtgcagtggagggggcaagctttgctccttcagttctgcttgctcccaagcccctgacccgctcggacttactgcctgaccttggaatcgtccctatatcagggcctgagtgacctccttctgcaaagtggcagggaccggcagagctctacaggcctgcagcccctaagtgcaaacacagcatgggtccagaagacgtggtctagaccagggctgctctttccacttgccctgtgttctttccccaggggcatgactgtcgccacacgcctctgtatatatgtctctttggagttggaatttcattatatgttaagaaaataaaggaaaatgacttgtaaggtc

SEQ ID NO: 6 - protein, 269 aa, Homo sapiens
MASEFKKKLFWRAVVAEFLATTLFVFISIGSALGFKYPVGNNQTAVQDNVKVSLAFGLSIATLAQSVGHISGAHLNPAVTLGLLLSCQISIFRALMYIIAQCVGAIVATAILSGITSSLTGNSLGRNDLADGVNSGQGLGIEIIGTLQLVLCVLATTDRRRRDLGGSAPLAIGLSVALGHLLAIDYTGCGINPARSFGSAVITHNFSNHWIFWVGPFIGGALAVLIYDFILAPRSSDLTDRVKVWTSGQVEEYDLDADDINSRVEMKPK

SEQ ID NO: 7 - DNA, 32 bp, Artificial Sequence
  primer AQY11-FP (ANT108)
gcgaattcttaactataacatgtcttcgaacg

SEQ ID NO: 8 - DNA, 32 bp, Artificial Sequence
  AQY11-RP (ANT109)
ccgaagcttaaaaacactaattacctcagtag

SEQ ID NO: 9 - DNA, 30 bp, Artificial Sequence
  AQY21-FP (ANT110)
gcgaattcatgtctaacgaatctaacgacc

SEQ ID NO: 10 - DNA, 31 bp, Artificial Sequence
  AQY21-RP (ANT111)
cgggatccgagcagactactctcagtcttcc

SEQ ID NO: 11 - DNA, 27 bp, Artificial Sequence
  AQY22-FP (ANT106)
atgaattcatgtctaacgaatctaacg

SEQ ID NO: 12 - DNA, 28 bp, Artificial Sequence
  AQY22-RP (ANT107)
atcccggggtcttccttcttttgacctg

SEQ ID NO: 13 - DNA, 18 bp, Artificial Sequence
  TPIprom-FW
cctacgttagtgtgagcg

SEQ ID NO: 14 - DNA, 18 bp, Artificial Sequence
  TPIprom-RW
cgctcacactaacgtagg

SEQ ID NO: 15 - DNA, 18 bp, Artificial Sequence
  KanFW
ggatgtatgggctaaatg

SEQ ID NO: 16 - DNA, 18 bp, Artificial Sequence
  KanRW
cctcgacatcatctgccc

SEQ ID NO: 17 - DNA, 64 bp, Artificial Sequence
  EANT1
agacgatgtctaataaatccggtacttctttacttgcaattaattactaagcggcgcctgtgtt

SEQ ID NO: 18 - DNA, 62 bp, Artificial Sequence
  EANT2
taaattaaactacgatgggagcgttatgccaaaaaagataaaattctgagcagactactctc

SEQ ID NO: 19 - DNA, 17 bp, Artificial Sequence
  ANT114
gcggacatcgatgcagg

SEQ ID NO: 20 - DNA, 20 bp, Artificial Sequence
  ANT115
ggaaagaatggatagtggta